(19) United States
(12) Patent Application Publication
(10) Pub. No.: US 2007/0092938 A1
(43) Pub. Date: Apr. 26, 2007

(54) Diagnostics for sars virus

(75) Inventors: Jimmy Kwang, Singapore (SG); Ai Ee Ling, Singapore (SG); Eng Eong Ooi, Singapore (SG); Hiok Hee Chng, Singapore (SG)

Correspondence Address: ROTHWELL, FIGG, ERNST & MANBECK, P.C., 1425 K STREET, N.W., SUITE 800, WASHINGTON, DC, 20005, US

(73) Assignee: Temasek Life Sciences Laboratory, Singapore (SG)

(21) Appl. No.: 10/564,617

(22) PCT Filed: Feb. 4, 2004
(86) PCT No.: PCT/US04/03307

§ 371 Date: Dec. 26, 2006

Related U.S. Application Data
(60) Provisional application No. 60/486,918, filed on Jul. 15, 2003.

(51) Int. Cl. C12Q 1/68; C12P 21/06

(52) U.S. Cl. 435/69.1; 435/6

(57) ABSTRACT

This invention relates to Severe Acute Respiratory Syndrome associated coronavirus (SARS virus) isolated and recombinant proteins, in particular the nucleocapsid (N) protein and spike (S) protein, as well as fragments thereof and their use in the diagnosis, treatment and prevention of Severe Acute Respiratory Syndrome (SARS). The proteins and fragments carry epitopes that are specific for the SARS virus. Thus, detection methods based on these proteins or fragments as well as the monoclonal antibodies against these proteins or fragments are specific for the SARS virus.

### CROSS-REFERENCE TO RELATED APPLICATION

The present application is related to and claims priority under 35 U.S.C. §119(e) to U.S. provisional patent application Ser. No. 60/486,918, filed Jul. 15, 2003, the entire content of which in incorporated herein by reference.

### FIELD OF THE INVENTION

The present invention relates to recombinantly expressed proteins from the SARS associated coronavirus (SARS virus), in particular nucleocapsid (N) protein and spike (S) protein, as well as fragments thereof and their use in diagnosis and of Severe Acute Respiratory Syndrome (SARS). The present invention also relates to antibodies, in particular monoclonal antibodies, against such recombinant proteins from the SARS virus and fragments thereof.

### BACKGROUND AND RECENT DEVELOPMENTS IN SARS RESEARCH

Throughout this application, various publications are referenced. Disclosures of these publications in their entireties are hereby incorporated by reference into this application.

In February 2003, a physician from Guangdong Province, China, fell ill while staying in a hotel in Hong Kong. Twelve other guests of the hotel fell ill, subsequently traveled and spread a disease which would come to be known as Severe Acute Respiratory Syndrome (SARS) to Vietnam, Singapore, Canada, Ireland, and the United States. As of Apr. 17, 2003 there had been 3389 cases and 165 deaths reported in 27 countries (1). In May 31, 2003 764 deaths and 8360 affected individuals were reported (2).

Several laboratories responded to the outbreak of SARS by quickly isolating a novel coronavirus (3, 4, 5, 6). On Apr. 16, 2003, the World Health Organization (WHO) announced that a new pathogen, a member of the coronavirus family not seen before in humans, is the cause of the Severe Acute Respiratory Syndrome. This new member of the coronavirus family is now known as the SARS virus or SARS coronavirus.

Coronavirus genomes consist of a single stranded (+) sense RNA and are approximately 27 kb to 30 kb long (7, 8). The genome of the SARS virus known as Tor2 is 29,751 bases long and has been fully sequenced (8).

The viral (+) RNA functions directly as mRNA. The 5′ 20 kb segment of the genome is translated first to produce a virus polymerase, which then produces a full length (−) sense RNA strand. This (−) sense RNA strand is used as a template to produce mRNA as a nested set of transcripts, all with an identical non-translated 5′ end. Each mRNA is monocistronic and has internal ribosomal binding sites (IRBS) (9). The genomic organization of SARS coronavirus is typical of coronavirus, with the characteristic gene order (replicase, S (spike), E (envelope), M (membrane) and N (nucleocapsid)). The three main structural proteins of the SARS virus are the N (nucleocapsid) protein, which binds to a defined packaging signal on newly synthesized viral (+) RNA to form nucleocapsid (NC), the M (matrix) protein, which is required for viral budding, and the S (spike) protein, oligomers of which form spikes in the envelope of the virus, which in turn bind to receptors on host cells and fuse the viral envelope with host cell membranes (8). The N protein also has a nuclear function, which might play a role in the pathogenesis of the SARS virus. In particular, the N protein of many coronaviruses, such as that of IBV (infectious bronchitis virus), is highly conserved among each group of coronaviruses, is immunogenic and abundantly expressed during infection. The N protein has become the target gene used for developing PCR for diagnostic purposes (10, 11, 12). For the development of an immunological diagnostic, the C terminus of the N protein is of particular interest (13, 14, 15).

Although human coronaviruses cause up to 30 percent of colds, they rarely cause lower respiratory tract disease. In contrast, animal coronaviruses are known to cause severe symptoms in animals (16). It has been speculated that the SARS virus originated in animals and mutated or recombined to permit it to infect humans. This theory is supported by preliminary evidence that suggests that antibodies to the isolates of the SARS virus are absent in those not infected with the virus (17). Recent studies suggest a pig origin.

SARS infections have been confirmed by detection of SARS RNA via PCR or via RT-PCR. PCR, while determining whether or not virus RNA is present in a sample, does not provide information as to whether a sample is infectious. Also, stringent laboratory protocols need to be adhered to avoid cross contamination of samples (18). Whether a sample contains infectious virus can be determined by inoculating suitable cell cultures, such as Vero cells, with a patient specimen. Generally, such cell cultures are generally very demanding and require biosafety levels (BSL) 3 facilities (19).

Two detection methods for SARS which are based on the presence of antibodies in the serum of a patient are enzyme linked immunoabsorbent assay (ELISA) and immunofluorescence assay (IFA). IFA generally involves the use of SARS infected cells which are fixed to a microscope slide. The antibodies in a serum sample bind to viral antigen and are made visible by immunofluorescent labeled secondary antibodies against human IgM or IgG or both. Generally, IFA is performed by laboratories with BSL-3 facilities (19). Original antigen production for ELISA also often involves the use of SARS infected cells.

Using immunological methods for the diagnosis of SARS bears the risk of false positives due to potential cross reactivity of the immunological detecting agent with, depending on the method employed, antibodies against or antigens of, non-SARS coronaviruses. There is also a risk of false negatives due to lack of universal reactivity of the immunological detecting agent with SARS antigen or antibody.

The SARS virus has been reported to share antigenic features with various group I coronaviruses. However, sequence analysis of the genes of the virus indicated that it is only distantly related to previously sequenced coronaviruses and does not fall within the three major coronavirus antigenic groups previously identified (17, 20, see also Examples: Homology Analysis).

Immunofluorescence staining revealed reactivity of the SARS virus with group I corona virus polyclonal antibody. Immunohistochemical assays with various antibodies reactive with coronavirus from antigenic group I, including porcine transmissible gastroenteritis virus, with an immune serum specimen from a patient with SARS have shown to have strong cytoplasmic and membranous staining effects in infected cells. However, the SARS virus could not be detected with an extensive panel of antibodies against coronaviruses representative of the three antigenic groups (17).

It would be highly desirable to be able to specifically recognize SARS virus in a serum by detecting specific antibodies against the virus. It also would be desirable to be able to recognize SARS virus via antibodies that can react with specific epitopes of the SARS virus. There is also a need for detection methods that are specific, easy to use and provide results quickly. There is furthermore a need for a detection method that can detect a SARS infection soon after the onset of symptoms. There is also a need for a detection method that requires no or relatively low BSL (biosafety level) facilities, such as BSL-2 or BSL-1 facilities.

### SUMMARY OF THE INVENTION

The invention is, according to a first aspect, a diagnostic method for detecting in a biological sample an antibody that binds to at least one epitope of a SARS virus. This method comprises contacting a biological sample with at least one isolated SARS virus protein or at least one fragment of the isolated SARS virus protein comprising at least one epitope of the SARS virus, and detecting the formation of an antigen-antibody complex between the virus protein or the fragment and an antibody present in the biological sample.

The at least one isolated SARS virus protein is, in one embodiment of this first and other aspects of the present invention, an N or S protein. In another embodiment of this first and other aspects of the present invention, the at least one fragment of the isolated SARS virus protein is between about 65 to about 423 amino acids long. The fragment may also be between about 65 and about 300 or between about 65 and about 200 amino acids long. A fragment of the N or S protein of the isolated SARS virus protein may be one of the fragments identified herein as N195, N210, N170, N71, N80A, N80B, N74, Fa, Fb, Fc, Fd, Fe, Ga, Gb, G1, G2, G3, G4, G5, G6, G7, G8, G9, G10, G1, G12, G13, G14, G15, G16, G17, G18 from SARS virus strain SIN 2774, a fragment substantially corresponding to said fragment(s), or mixtures thereof. In a preferred embodiment, the fragment is the fragment identified herein as N195 or Fc from SARS virus strain SIN 2774, a fragment having substantially the same amino acid sequence as said fragment(s), a fragment substantially corresponding to said fragment(s), or mixtures thereof.

The formation of antigen-antibody complex is detected, in one embodiment of this first and other aspects of the present invention, by radioimmunoassay (RIA), enzyme linked immunosorbent assay (ELISA), immunofluorescence assay (IFA), dot blot or western blot. In particular, the formation may be detected by ELISA, dot blot or western blot.

The invention is, according to a second aspect of the present invention, an in vitro diagnostic kit for detecting in a biological sample an antibody against a SARS virus. The diagnostic kit comprises at least one isolated SARS virus protein, or at least one fragment of the isolated SARS virus protein comprising at least one epitope of the SARS virus, reagents for detecting the formation of antigen-antibody complex between the at least one isolated SARS virus protein or fragment thereof and at least one antibody present in the biological sample, wherein the at least one isolated protein or fragment thereof and the reagents are present in an amount sufficient to detect the formation of antigen-antibody complex.

The invention is, according to a third aspect of the present invention, a method for determining an epitope specific for the SARS virus. This method comprises providing at least one fragment of at least one protein of the SARS virus, wherein the at least one fragment is at least 65 amino acids long, reacting the at least one fragment with (a) at least one serum sample from a SARS positive human, and with (b) at least one serum sample from a coronavirus positive, SARS negative, human or non-human animal, detecting fragment-antibody complexes formed from the reactions of the at least one fragment with (a) and (b), and selecting one or more fragments comprising epitopes specific for the SARS virus by selecting fragments that form fragment-antibody complexes with (a), but not with (b). In one embodiment of this third aspect of the invention, the fragment is reacted with sera from at least 5 SARS positive humans. In another embodiment of this third aspect of the invention, the at least one serum sample from a coronavirus positive, SARS negative, human or non-human animal, is chicken serum against IBV or pig serum against TGE.

The invention is, according to a fourth aspect of the present invention, a method for inducing an immune response against SARS virus in a non-human animal or human. The method comprises selecting at least one isolated SARS virus protein or at least one fragment thereof competent to induce a protective immune response in a non-human animal against a SARS virus, and administering to a non-human animal or human an effective amount of the SARS virus protein(s) or fragment(s) thereof sufficient to induce an immune response against the SARS virus. In one embodiment of this fourth aspect of the invention, the non-human animal is a guinea pig, swine, mouse, rat, cat or a bird. In another embodiment of this fourth aspect of the invention, the antibodies are isolated from the non-human animal and are compared to antibodies from humans recovered from a SARS infection.

The invention is according to fifth and sixth aspects of the present invention, respectively, a diagnostic method for detecting the presence in at least one biological sample of at least one antibody against a SARS virus. These methods comprise contacting a biological sample with one or more peptides comprising at least about 65 contiguous amino acid residues of SEQ ID No. 2 or SEQ ID No. 4, or one or more peptides comprising at least about 65 amino acid residues and having at least about 90% sequence identity with a contiguous number of amino acid residues of SEQ ID No. 2 or SEQ ID No. 4 having about equal length as said one or more peptides, wherein said one or more peptides comprise at least one epitope of a SARS virus, and detecting whether an antigen-antibody complex has formed between said one or more peptides and antibodies present in said biological sample. SEQ ID No. 2 is the full length amino acid sequence of the N protein of SARS virus strain SIN 2774, SEQ ID No. 4 is the full length amino acid sequence of the S protein of SARS virus strain SIN 2774. In one embodiment of said fifth aspect of the present invention said one or more peptides have at least about 95% sequence identity with a contiguous number of amino acid residues of SEQ ID No. 6 having about equal length as said one or more peptides. In one embodiment of said sixth aspect of the present invention said one or more peptides have at least about 95% sequence identity with a contiguous number of amino acid residues of SEQ ID No. 8 having about equal length as said one or more peptides.

SEQ ID No. 6 is the amino acid sequence of fragment N195 of SARS virus strain SIN 2774, SEQ ID No. 8 is the full length amino acid sequence of fragment Fc of SARS virus strain SIN 2774.

In seventh and eighth aspects, respectively, the present invention is an isolated and purified nucleic acid comprising an polynucleotide comprising at least about 195 contiguous nucleotides of SEQ ID No. 1 or SEQ ID No. 3, or at least one polynucleotide comprising at least about 195 contiguous nucleotides which have at least about 75% homology with a contiguous number of nucleotides of SEQ ID No. 1 or SEQ ID No. 3 having about equal length as said at least one polynucleotide, wherein said polynucleotide encodes a peptide that is adapted to detect anti-SARS-antibody in a sample. In one embodiment of the ninth aspect of the invention, the polynucleotide hybridizes under stringent conditions with a contiguous number of nucleotides of SEQ ID No. 5 having about equal length as said at least one polynucleotide. In one embodiment of the tenth aspect of the invention, the polynucleotide hybridizes under stringent conditions with a contiguous number of nucleotides of SEQ ID No. 7 having about equal length as said at least one polynucleotide. SEQ ID No. 5 is the nucleic acid sequence encoding fragment N195, SEQ ID No. 7 is the nucleic acid sequence encoding fragment Fc.

In a ninth aspect, the present invention is a method for producing a monoclonal antibody against at least one SARS protein. The method comprises (a) injecting at least one antigenic fragment of the SARS protein into a non-human animal, (b) isolating at least one spleen cell from the non-human animal, (c) fusing the spleen cell with a myeloma cell, (d) screening the resulting hybridoma cells with the at least one SARS protein for the production of monoclonal antibody against the at least one SARS protein, and (e) selecting at least one hybridoma cell producing the monoclonal antibody.

In a tenth aspect, the present invention is a diagnostic method for detecting a SARS virus in at least one biological sample. The diagnostic method comprises (a) contacting the at least one biological sample with at least one monoclonal antibody against a SARS virus protein, wherein said at least one monoclonal antibody derived from a non-human animal injected with an antigenic fragment of a SARS virus protein, and (b) detecting the formation of a complex between the monoclonal antibody and said SARS virus.

In eleventh and twelfth aspects, respectively, the present invention is a diagnostic method for detecting a SARS virus in at least one biological sample. The methods comprise (a) contacting the at least one biological sample with at least one monoclonal antibody against a SARS virus protein, wherein said at least one monoclonal antibody is derived from a non-human animal injected with an antigenic peptide comprising at least about 65 contiguous amino acid residues of SEQ ID No. 2 or SEQ ID No. 4, respectively, or an antigenic peptide comprising at least about 65 amino acid residues and having at least about 90% sequence identity with a contiguous number of amino acid residues of SEQ ID No. 2 or SEQ ID No. 4, respectively, and having about equal length as said antigenic peptide, and (b) detecting the formation of a complex between the monoclonal antibody and the SARS virus.

The invention also includes antibodies against the proteins and peptides described above and diagnostic kits comprising such antibodies.

### BRIEF DESCRIPTION OF THE DRAWINGS

FIG. 1 is a diagram illustrating fragments of the 1269 bp nucleocapsid protein from the SARS virus strain SIN2774, namely N210, N195, N170, N71, N80A, N80B and N74.

FIGS. 2a and 2b are SDS-PAGE gels to analyze the expression of N210, N195, N170, N71, N80A and N74 as GST fusion proteins after induction. The left lanes show a molecular marker and lanes “U” are uninduced controls.

FIGS. 3a and 3b are SDS-PAGE gels showing the N210, N195, N170, N71, N80A and N74 as GST fusion proteins after protein purification. The respective left lanes show molecular weight markers.

FIG. 4a is a western blot showing in lane 1, a reaction of N195 with serum from SARS positive humans and in the remaining lanes, lack of a reaction of N195 with different sera, namely in lane 2 with serum from SARS negative humans, in lane 3 with serum from TGE positive pigs, in lane 4 with serum from TGE negative pigs, in lane 5 with serum from IBV positive chicken and in lane 6 with serum from IBV negative chicken.

FIG. 4b is a western blot showing in lane 1, a reaction of N210 with serum from SARS positive humans and in the remaining lanes, lack of a reaction of N210 with different sera, namely in lane 2 with serum from SARS negative humans, in lane 3 with serum from TGE positive pigs, in lane 4 with serum from TGE negative pigs, in lane 5 with serum from IBV positive chicken and in lane 6 with serum from IBV negative chicken.

FIGS. 4c-4f are western blots of N195 fragments reacted with different serum samples from cats infected with cat coronavirus (4c), dogs infected with dog coronavirus (4d), chicken infected with avian coronavirus (4e), pigs infected with porcine coronavirus (4f). Lanes “+” indicate positive controls, the remaining numbered lanes indicate different sera from the respective animal specie. All of the numbered lanes show lack of reaction with N195.

FIG. 5a is a western blot using anti human IgG showing reaction of N195 with 10 sera from SARS positive humans. Lanes 11 and 12 show a negative and positive control, respectively.

FIG. 5b is a western blot showing the absence of a reaction of N195 with 10 sera from SARS negative humans. Lanes 11 and 12 show a negative and positive control, respectively.

FIG. 6 shows the results of an ELISA testing for IgG antibodies against SARS virus using a single recombinant N195 fragment as the coating antigen. “Negative” indicates the results with SARS negative serum samples, “Positive” indicates the results with SARS positive serum samples.

FIG. 7 is a diagram illustrating fragments of the 1255 aa Spike protein from the SARS virus strain SIN2774, namely fragments Fa, Fb, Fc, Fd, Fe (1a), Ga, Gb (1b), and G1 to G18 (1c).

FIGS. 8a and 8b are SDS-PAGE gels showing the expression of fragments G1 to G18. Lanes M are molecular weight markers, lane “U” is an uninduced control, lane “GST” is a GST control.

FIGS. 9a and 9b are SDS-PAGE gels illustrating the purified fragments of G1-G10. “U” indicate lanes showing uninduced controls, the left lanes show molecular weight markers.

FIG. 10 is a western blot illustrating the expression of fragments G1-G18 by anti-GST antibody. Lane “GST” shows a GST control, lane “M” shows a molecular weight marker. Table 7 shows reactivity of the 18 S protein fragments against 10 SARS-positive serum samples.

FIG. 11 is a western blot of Fa to Fe spike protein fragments visualized with anti-His6 antibody. Table 8 shows reactivities of the 10 SARS-positive serum samples with fragments Fa-Fe of S protein expressed from insect cells.

FIG. 12 is a western blot of Ga and Gb protein fragments visualized with anti-GST antibody.

### DETAILED DESCRIPTION OF THE PREFERRED EMBODIMENT

The present invention provides for isolated and recombinantly expressed protein of SARS virus, in particular nucleocapsid (N) protein and isolated (S) protein, and fragments thereof for the detection of SARS specific antibodies in infected humans.

Definitions

A number of SARS virus strains and individual proteins of such strains have been isolated and fully identified (20, 24). Identification of further strains and individual proteins of such strains is in progress. It will be understood by the person skilled in the art that methods identified herein and products obtained by those methods can be performed/produced with a wide variety of SARS virus strains. Thus a “SARS virus” according to the present invention includes any SARS virus strain. While the examples have been performed with SARS virus strain 2774, the person skilled in the art will readily appreciate that those examples can be extrapolated to other SARS virus strains.

A “SARS virus protein” according to the present invention is any protein of any SARS virus strain or its functional equivalent as defined herein. Thus, the invention includes, but is not limited to, SARS polymerase, the S (spike) protein, the N (nucleocapsid) protein, the M (membrane) protein, the small envelope E protein and their functional equivalents.

A “fragment” of a SARS virus protein according to the present invention is a partial amino acid sequence of a SARS virus protein or a functional equivalent of such a fragment. A fragment is shorter than the complete virus protein and is preferably between about 65 and about 423 amino acids long, more preferably between about 65 and about 300 amino acids long, even more preferably between about 65 and about 200 amino acids long. Also, a fragment can be derived from either terminus of the virus protein or from an inner portion of the virus protein as described below. While a “fragment” of a SARS virus can generally be obtained from any SARS strain, preferred fragments are nucleocapsid protein fragment N195 and spike protein fragment Fc from strain SIN2774 and fragments from other strains substantially corresponding to these fragments, as defined herein. A fragment of a SARS virus protein also includes peptides having at least 65 contiguous amino acid residues having at least about 70%, at least about 80%, at least about 90%, preferably at least about 95%, more preferably at least 98% sequence identity with at least about 65 contiguous amino acid residues of SEQ ID No. 2, 4, 6 or 8 having about the same length as said peptides. Depending on the expression system chosen, the protein fragments may or may not be expressed in native glycosylated form.

A “functional equivalent” of a SARS virus protein or a fragment of such a protein according to the present invention is an amino acid sequence that has, e.g., one or more amino acid substitutions, internal deletions, additions or non native glycosylations, which, however, do not affect the protein's or the fragment's function according to the present invention, e.g., its ability to act as an antigen in an antigen-antibody complex and/or in its ability to induce an immune response by raising antibodies that can be used for the detection of the SARS virus.

A fragment that “corresponds substantially to” a fragment of a protein of SIN 2774 is a fragment that has substantially the same amino acid sequence and has substantially the same functionality as the specified fragment of SIN 2774. Such a fragment may be, but is not limited to, a fragment from another strain of SARS or a synthetic fragment. Any deviations in, e.g., amino acid numbers and/or sequence result, e.g., from the alternate origin of the fragment as will be readily recognized by the person skilled in the art. A fragment that has “substantially the same amino acid sequence” as a fragment of a protein of SIN 2774 typically has more than 90% amino acid identity with this fragment. Included in this definition are conservative amino acid substitutions.

“Epitope” as used herein refers to an antigenic determinant of a polypeptide. An epitope could comprise three amino acids in a spatial conformation which is unique to the epitope. Generally, an epitope consists of at least five such amino acids, and more usually consists of at least 8-10 such amino acids. Methods of determining the spatial conformation of such amino acids are known in the art.

“Antibodies” as used herein are polyclonal and/or monoclonal antibodies or fragments thereof, including recombinant antibody fragments, as well as immunologic binding equivalents thereof, which are capable of specifically binding to SARS virus protein and fragments thereof or to polynucleotide sequences encoding such protein or fragments thereof. The term “antibody” is used to refer to either a homogeneous molecular entity or a mixture such as a serum product made up of a plurality of different molecular entities. Recombinant antibody fragments may, e.g., be derived from a monoclonal antibody or may be isolated from libraries constructed from an immunized non-human animal.

“Sensitivity” as used herein in the context of testing a biological sample is the percentile of the number of true positive SARS samples divided by the total of the number of true positive SARS samples plus the number of false negative SARS samples (See Table 9 for an example).

“Specificity” as used herein in the context of testing a biological sample is the percentile of the number of true negative SARS samples divided by the total of the number of true negative SARS samples plus the number of false positive samples (See Table 9 for an example).

“Detection rate” as used herein in the context of antibodies specific for a SARS virus is the percentile of the number of SARS positive samples in which the antibody was detected divided by the total number of SARS positive samples tested. E.g. an IgM detection rate (rate for detection of IgM antibodies) of 56.8% of a sample of 44 SARS positive biological samples means that 25 out of the 44 samples tested positive for IgM antibodies. “Overall detection rate” as used herein refers to the virus detection obtained by detecting both IgM and IgG.

A “clinical sample” comprises biological samples from a random mix of patients, including patients with and without SARS and patients with SARS at varying stages and patients with other illnesses that, however, show symptoms as defined herein.

“Onset of symptoms” as used herein is the onset of fever and a cough.

A nucleic acid of the present invention has substantial identity with another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases. A protein or peptide of the present invention has substantial identity with another if, optimally aligned, there is an amino acid sequence identity of at least about 60% identity with an naturally-occurring protein or with a peptide derived therefrom, usually at least about 70% identity, more usually at least about 80% identity, preferably at least about 90% identity, and more preferably at least about 95% identity, and most preferably at least about 98% identity.

Identity means the degree of sequence relatedness between two polypeptide or two polynucleotides sequences as determined by the identity of the match between two strings of such sequences, such as the full and complete sequence. Identity can be readily calculated. While there exist a number of methods to measure identity between two polynucleotide or polypeptide sequences, the term “identity” is well known to skilled artisans (31-35). Methods commonly employed to determine identity between two sequences include, but are not limited to, those disclosed in Guide to Huge Computers (23). Preferred methods to determine identity are designed to give the largest match between the two sequences tested. Such methods are codified in computer programs. Preferred computer program methods to determine identity between two sequences include, but are not limited to, GCG (Genetics Computer Group, Madison Wis.) program package (36), BLASTP, BLASTN and FASTA (37-38). The well-known Smith Waterman algorithm may also be used to determine identity.

As an illustration, by a polynucleotide having a nucleotide sequence having at least, for example, 95% “identity” to a reference nucleotide sequence means that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

Alternatively, substantial homology or (similarity) exists when a nucleic acid or fragment thereof will hybridize to another nucleic acid (or a complementary strand thereof under selective hybridization conditions, to a strand, or to its complement. Selectivity of hybridization exists when hybridization which is substantially more selective than total lack of specificity occurs. Typically, selective hybridization will occur when there is at least about 55% homology over a stretch of at least about 14 nucleotides, preferably at least about 65%, more preferably at least about 75%, and most preferably at least about 90%. The length of homology comparison, as described, may be over longer stretches, and in certain embodiments will often be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter. The stringency conditions are dependent on the length of the nucleic acid and the base composition of the nucleic acid, and can be determined by techniques well known in the art. See, e.g., Asubel, 1992; Wetmur and Davidson, 1968.

Thus, as herein used, the term “stringent conditions” means hybridization will occur only if there is at least 95% and preferably at least 97% identity between the sequences. Such hybridization techniques are well known to those of skill in the art. Stringent hybridization conditions are as defined above or, alternatively, conditions under overnight incubation at 42° C. in a solution comprising: 50% formamide, 5×SSC (150 mM NaCl, 15 mM trisodium citrate), 50 mM sodium phosphate (pH7.6), 5× Denhardt's solution, 10% dextran sulfate, and 20 μg/ml denatured, sheared salmon sperm DNA, followed by washing the filters in 0.1×SSC at about 65° C.

In one embodiment, the present invention relates to the detection of SARS virus in a serum sample either by detecting antibodies against SARS in such a serum sample or by detecting epitopes of the SARS virus.

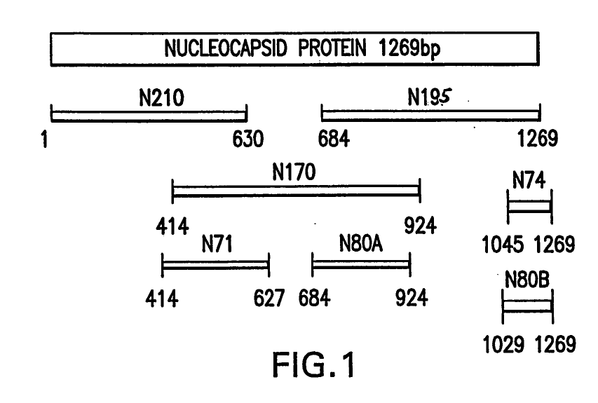

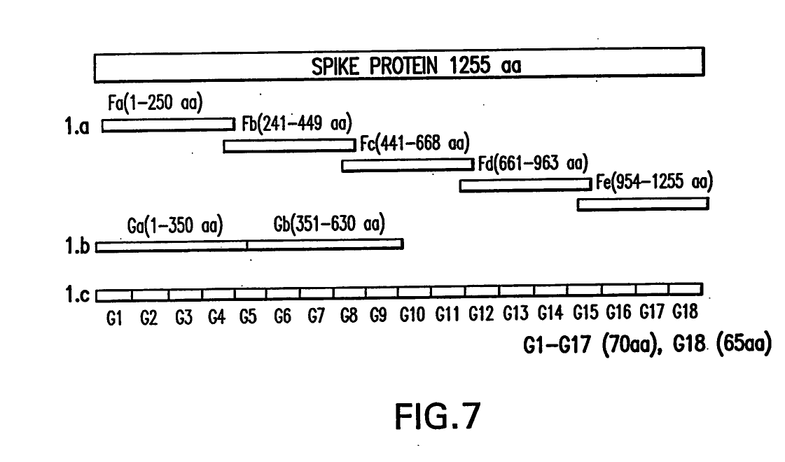

One preferred embodiment comprises a diagnostic method or a diagnostic kit (hereinafter commonly referred to as a “diagnostic”) that allows for the detection of specific antibodies against the SARS virus via complex formation with at least one fragment of a SARS protein. One way, although not the only way contemplated by the present invention, to increase the specificity of detection is to precisely map the location of one or more epitopes on a SARS virus protein. To achieve this goal, progressively smaller fragments of SARS virus protein are tested. Small fragment size is preferable, though not required, for proteins that have a high mutation rate. Another preferred embodiment uses highly conserved proteins and fragments thereof. Yet another preferred embodiment comprises a diagnostic that comprises more than one fragment of a SARS virus protein and that allows for the detection of specific antibodies against those fragments. These fragments of SARS virus protein may, but are not required to, contain epitopes that can react with sera from different infections stages of the SARS virus, e.g. an early and a late stage. However epitopes that can react with sera from different infection stages may also be located on a single fragment. Another preferred embodiment comprises a diagnostic that allows for the detection of specific antibodies against a SARS virus via complex formation with fragment N195 or N210 of the N protein of the SARS virus strain SIN 2774 or with combinations thereof (FIG. 1; Table 2) or with substantially corresponding fragments of other SARS virus strains. Yet another preferred embodiment comprises a diagnostic that allows for the detection of specific antibodies against the SARS virus via complex formation with at least one fragment of the S protein. Such S protein fragments are preferably one or more of fragments Fc and G9 of the S protein of SARS virus strain SIN 2774 (FIG. 7; Tables 3 and 4) or substantially corresponding fragments of other SARS virus strains. Combinations of SARS virus protein fragments, such as N195 and Fc, or full length proteins, such as the N and S protein, are also within the scope of the present invention.

In a preferred embodiment, fragments that display little or no crossreactivity with other commonly encountered coronaviruses are used. In another preferred embodiment, fragments are selected that display little or no non-specific reaction with sera from patients having an autoimmune disease. In another preferred embodiment, fragments are selected that can be produced in high quantities, that is, have a high protein yield. In another preferred embodiment, fragments are selected that can be easily purified. In certain embodiments, the fragment(s) are synthesized. In another embodiment, the fragment(s) are immunodominant. In yet another preferred embodiment, the fragment(s) have a high detection rate for IgM and/or IgG.

Another preferred embodiment comprises a diagnostic that allows for the detection of SARS virus via complex formation between an epitope of the SARS virus and at least one specific antibody against this epitope. Such an antibody can be raised by administering to a non-human animal, such as mouse, an immunogenic composition comprising an immunoefficient amount of at least one isolated protein of a SARS protein or a fragment thereof. Such an antibody can be directly or indirectly labeled and can be a monoclonal antibody.

The existence of antigen-antibody binding can be detected via methods well known in the art. In western blotting, one preferred method according to the present invention, fragments of a protein are transferred from the gel to a stable support such as a nitrocellulose membrane. The protein fragments can be reacted with sera from individuals infected with the SARS virus. This step is followed by a washing step that will remove unbound antibody, but retains antigen-antibody complexes. The antigen-antibody complexes then can be detected via anti-immunoglobulin antibodies which are labeled, e.g., with radioisotopes.

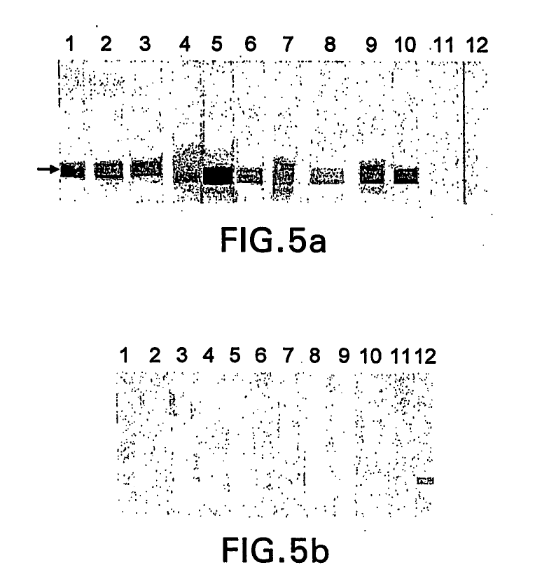

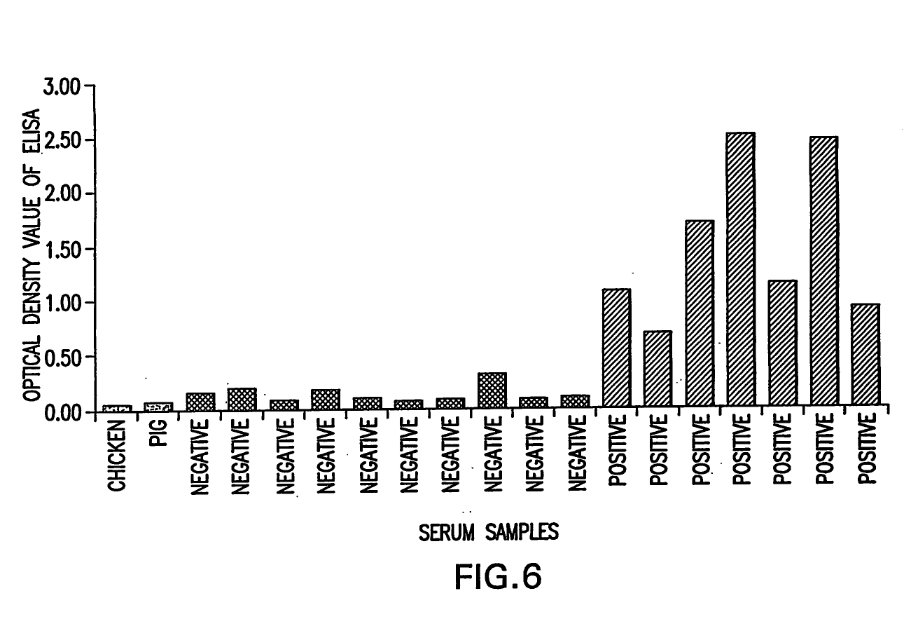

Use of a western blot allows detection of the binding of sera of SARS positive human to any antigen of the SARS virus. Such antigens include, but are not limited to, the virus polymerase(s), the S (spike) protein, the N (nucleocapsid) protein, the M (membrane) protein, the small envelope E protein and any fragment(s) of such proteins. FIGS. 5a and 5b show the specific binding of ten SARS positive sera from different patients with the N195 and N210 fragments of the nucleocapsid protein as well as one negative and positive control.

Other preferred detections methods include enzyme-linked immunosorbent assays (ELISA) and dot blotting. Both of these methods are relatively easy to use and are high throughput methods. ELISA, in particular, has achieved high acceptability with clinical personnel. ELISA is also highly sensitive. However, any other suitable method to detect antigen-antibody complexes such as, but not limited to, standardized radioimmunoassays (RIA) or immunofluorescence assays (IFA), also can be used.

Another preferred embodiment of the present invention comprises an IFA type detection method in which SARS proteins or fragments thereof, such as N195, are expressed in eukaryotic cells, such as insect cells, through recombinant viruses, such as insect viruses. In a preferred embodiment, fusion proteins of two or more immunodominant antigens from the same or different proteins of the SARS virus, such as N195 and Fc, are used for detecting the presence of SARS antibody in a sample. In one embodiment, the invention comprises a fusion protein having the N195 fragment at its N terminus and the Fc fragment at its C terminus. In another embodiment, the invention comprises a fusion protein having the Fc fragment at its N terminus and the N195 fragment at its C terminus. Such fusion proteins are, in one embodiment of the present invention, expressed in insect cells. Those insect cells are, in a preferred embodiment, fixed to an assay plate and reacted with the sera of a patient. SARS antibodies reacting with the fusions proteins can be visualized via a fluorescein labeled antibody. This IFA using proteins of SARS or fragments thereof is safer than a traditional IFA, as it does not require handling of whole live virus. The assay may be performed in laboratories having BSL 2 facilities, while a traditional IFA requires BSL 3 facilities. In a preferred embodiment, the inventive IFA has high sensitivity and specificity, which equals or exceeds the sensitivity and specificity of traditional IFAs using whole live SARS virus. In another embodiment, the IFA of the present invention is more sensitive in the detection of SARS than a western blot assay. In yet another embodiment, it requires less than 2 hours, more preferable 1.5 hours or less and even more preferably 1 hour or less, to complete the inventive assay.

Another preferred embodiment of the present invention comprises a detection method comprising antibodies, in particular monoclonal antibodies, against proteins of SARS such as the N protein or the S protein, in particular, against specific epitopes of those proteins. Monoclonal antibodies are, in a preferred embodiment, produced by injecting purified antigenic fragments of SARS protein, such as N195 or Fc, into mice and producing hybridoma cells by fusing immune spleen cells of injected mice with myeloma cells and selecting hybridoma cells that produce the appropriate monoclonal antibody. In a preferred embodiment, a biological sample from a subject suspected of being infected with a SARS virus is attached to a support, such as a solid support or a membrane, and SARS virus is detected via such a monoclonal antibody, which is directly labeled, e.g., radioactively (for a RIA), with a suitable fluorochrome, e.g. fluorescein isothiocyanate (FITC) or and enzyme (for an ELISA). In another embodiment, the monoclonal antibody is detected via a secondary labeled antibody. In yet another embodiment, the monoclonal antibody is attached to a support and a biological sample as defined below is added. SARS virus that binds to this monoclonal antibody may be detected via another labeled antibody against SARS virus.

Appropriate biological samples include, but are not limited to, mouth gargles, any biological fluids, virus isolates, tissue sections, wild and laboratory animal samples. The monoclonal antibody of the present invention may also be used, e.g., in competitive enzyme-linked immunosorbent assays (cELISAs) and direct double antibody sandwich enzyme-linked immunoabsorbent assays (DAS-ELISAs). However, as the person skilled in the art will appreciate, the monoclonal antibodies of the present invention may be used in many different assays to directly or indirectly detect the presence of a SARS virus in a biological sample. Also within the scope of the present invention are recombinant antibody fragments that can be grown in bacteria, e.g. E. coli.

In another preferred embodiment proteins or protein fragments are tested to determine whether or not a diagnostic method based on them has the desired detection rate for antibodies such as IgG and IgM, the desired overall detection rate, sensitivity and/or specificity. An appropriate test would be a blind test using a clinical sample. In such a clinical sample, sera from individuals infected with SARS generally, though not always, vary widely. Some sera will have been obtained from individuals who have recently been infected, others will have been obtained from individuals who have been infected for many weeks. Depending on the stage of the infection, antibody concentration and quality may vary. While the mean time of seroconversion for SARS coronavirus infections was reported to be 20 days (21, 22), sera from some patients have an uncommonly low number of detectable antibodies for extended periods of time. Also, the number of patients contained in such a sample will vary widely. In a preferred embodiment, the overall detection rate accomplished using a diagnostic method using particular protein(s) or fragment(s) thereof for such a clinical sample is more than 65%, more than 70%, more than 75%, more than 80%, more than 85%, more than 90%, more than 95% or 100%. In another preferred embodiment, the IgM detection rate for such a sample is more than 30%, more than 35%, more than 40%, more than 45%, more than 50%, more than 55% or more than 60%. In another preferred embodiment, the IgG detection rate for such a sample is more than 60%, more than 65%, more than 70%, more than 75%, more than 80%, more than 85% or more than 90%. In another preferred embodiment, the sensitivity of a diagnostic method using a particular protein or fragment thereof in the context of a clinical sample is more than 80%, more than 85%, more than 90%, more than 95%, more than 98%, more than 99% or 100%. In another preferred embodiment, the specificity of a diagnostic method with such a sample is more than 80%, more than 85%, more than 90%, more than 95%, more than 98%, more than 99% or 100%.

In one preferred embodiment, a diagnostic according to the present invention is able to detect IgG at a dilution of about 1:100, about 1:800, about 1:900, about 1:1000, about 1:1100 up to about 1:1200. In another preferred embodiment, a diagnostic according to the present invention is able to detect IgM at a dilution of about 1:50, about 1:100, about 1:500 up to about 1:1000. In a particularly preferred embodiment, a western blot used in the present invention is able to detect IgG at a dilution of about 1:800. In another particularly preferred embodiment, a western blot used in the present invention is able to detect IgM at a dilution of about 1:100.

In a preferred embodiment, a diagnostic according to the present invention will be able to detect a wide array of stages of a SARS infection. In another preferred embodiment, a diagnostic will be able to detect early stages of a SARS infection. In another preferred embodiment, a diagnostic will be able to detect early stages of infection by being able to detect IgM. In another preferred embodiment, an diagnostic will be able to detect early stages of infection by being able to detect very low concentrations of antibodies. Accordingly, in a preferred embodiment the diagnostic method is adapted to detect antibodies against a SARS virus less than about 50 days after the onset of symptoms, preferably less than about 40, less than about 30, less than about 25, less than about 20, less than about 15, less than about 12, less than about 10, less than about 9, less than about 8, less than about 7, less than about 6, less than about 5, less than about 4, less than about 3, less than about 2, less than 1 day after the onset of symptoms.

In a preferred embodiment, the detection method of the present invention is easy to use. In another preferred embodiment, the detection method of the present invention can be performed in laboratories having no biosafety level (BSL) facilities or facilities with a BSL of less than 3, more preferably of less than 2.

In order to produce high amounts of SARS protein and fragments thereof, the DNA fragments from genomic RNA can be produced by RT-PCR. The appropriate PCR primers can include restriction enzyme cleavage sites. After purification, the PCR products can be digested with the suitable restriction enzymes and cloned into suitable expression vectors, preferably, under the control of a strong promoter. The vectors then can be transformed into an appropriate host cell. Positive clones can be identified by PCR screening and further confirmed by enzymatic cut and sequence analysis. In one embodiment, the N protein and/or S-protein are expressed as fusion proteins, such as GST fusion proteins, with subsequent separation of the GST protein from the protein fragment, among others, to eliminate the cross reaction in human serum detection (12). The so produced proteins/fragments then can be tested for their suitability as antigens for a diagnostic.

The uses of the SARS virus proteins and fragments thereof according to the present invention that are described above are those which presently appear most attractive. However, the foregoing disclosures of embodiments of the invention and uses therefor have been given merely for purposes of illustration and not to limit is the invention. Thus, the invention should be considered to include all embodiments falling within the scope of the claims following the Example section and any equivalents thereof.

The following examples refer to nucleotide acid sequences, proteins and peptides isolated from SARS strain SIN2774 (25). However, the presently claimed invention encompasses nucleotide acid sequences, proteins and peptides isolated from any SARS strain and the modification described herein. In light of the description provided herein one of ordinary skill in the art can practice the invention to its fullest extent. The following example, therefore, is merely illustrative and should not be construed to limit in any way the invention as set forth in the claims which follow.

### EXAMPLES

The genomic RNA sequences of SIN2774 referred to and used in the following examples is accessible via NCBI Entrez Accession No. AY283798 (25) (SEQ ID No. 9). The entire sequence of SIN2774, accessible via NCBI Entrez Accession No. AY283798, is incorporated into this application by reference. Human sera used in the experiments described herein were collected from various institutions listed in Table 1. Each patient listed in the Table had a confirmed clinical diagnosis. All human sera were inactivated at 56° C. for 30 mins.

TABLE 1

| Serum group | No. | Origin of serum samples |
|---|---|---|
| Convalescent SARS | 6 | National Environment Agency, |
| patient sera* | | Singapore; Center for Disease Control, |
| | | Guangzhou, China |
| Confirmed SARS | 27 | Singapore General Hospital, Singapore; |
| patient sera* | | Tan Tock Seng Hospital, Singapore |
| “SARS positive | 33 = Sum of the above sera | |
| sera” | | |
| Normal Human sera | 66 | Singapore General Hospital, Singapore; |
| “SARS negative | | Tan Tock Seng Hospital, Singapore; |
| sera” | | volunteered blood donors |
| Clinically | 274 | Singapore General Hospital, Singapore; |
| blinded sera | | Tan Tock Seng Hospital, Singapore |

*All patients satisfied the WHO definition of SARS (22). These sera samples were collected from 4-49 days post fever, mean day of onset (mean 18.79; median 14.5; SD 11.95; SEM 2.26).

Four infectious bronchitis virus (IBV) infected chicken sera and 7 transmissible gastroenteritis viruses (TGEV) infected swine sera were available. 12 canine coronavirus vaccinated dog sera from Taiwan were used to check cross reaction. 10 stray dog sera and 10 stray cat sera provided by Agri-food and Veterinary Authority of Singapore were used as well.

Homology Analyses

The homology of the SARS gene encoding the N protein was compared to the genes encoding N protein of other human coronaviruses and other animal coronaviruses using bioinformatic methods.

Sequences of the gene encoding N protein in the SARS coronavirus were found to have 26-32% homology with the genes for the N protein of various animal coronaviruses.

Determination of Cross Reactivity of Full Length Nucleocapsid (N) Protein with Related coronaviruses

Full length N protein (SEQ ID No. 2) was expressed as discussed below. The protein was reacted with sera from chicken and pig immunized with avian and porcine coronavirus, respectively. Cross reaction was observed with sera from both chicken and pig.

Nucleocapsid (N) Protein Fragments

Seven partially overlapping fragments of the 1269 bp N protein sequence of SIN2774 (NCBI Entrez Accession No. AY283798) were created as discussed below. These fragments are shown in FIG. 1. The base pairs that constitute the respective fragments are also listed in Table 2.

TABLE 2

| N protein fragment number | base pairs of N protein |
|---|---|
| N210 | 1-630 |
| N195 (SEQ ID No. 6) | 684-1269 (SEQ ID No. 5) |
| N170 | 414-924 |
| N71 | 414-627 |
| N80A | 684-924 |
| N80B | 1029-1269 |
| N74 | 1045-1269 |

Spike (S) Protein Fragments

Preliminary studies of infectious bronchitis virus (IBV) and transmissible gastroenteritis virus (TGEV) revealed that neutralizing epitopes of those coronaviruses were located at the N-terminus of the spike proteins. Accordingly, some precedence was given in the search for epitopes to the N terminus of the S protein of the SARS virus. However, other parts of the S protein were also investigated.

Two sets of fragments of the 1255 amino acid long S protein (SEQ ID No. 4) of strain SIN2774 were created as discussed below and are shown in Tables 3 and 4 and depicted in FIG. 7. As can be seen fragments Ga, Gb, Fa and Fb originate from the N terminus of the protein.

TABLE 3

| Name of Fragment of S protein | Corresponding aas of S protein |
|---|---|
| Fa | 1-250 |
| Fb | 241-449 |
| Fc (SEQ ID No. 8) | 441-668 (SEQ ID No. 8)* |
| Fd | 661-963 |
| Fe | 954-1255 |

*SEQ ID No. 7 represents the corresponding DNA sequence; SEQ ID No. 3 represents DNA encoding the full S protein.

TABLE 4

| Name of Fragment of S protein | Corresponding aas of S protein |
|---|---|
| Ga | 1-350 |
| Gb | 351-630 |

A third set of 18 fragments of the S protein was created and labeled G1 to G18. Each of these fragments constituted a peptide of 70 consecutive amino acids of the spike protein, wherein G1 consisted of amino acid residues 1-70 of the spike protein, G2 consisted of amino acid residues 71-140 of the spike protein etc. G18 consists of the C terminal 65 amino acids. See 1.c in FIG. 7.

Production of Proteins and Fragments

Molecular Cloning

The supernatant of SARS coronavirsus (SIN2774) cell culture was inactivated before it was used for RNA extraction. Viral RNA was extracted using Trizol reagents (Gibco, New York) and was reverse transcribed to produce DNA.

The full length and six fragments of the N protein was amplified using standard polymerase chain reaction (PCR; 94° C., 4 mins.; followed 30 circles of 94° C., 1 min.; 55° C., 1 min.; 72° C., 1 min). BamHI and SalI cleavage sites were included in the forward and reverse primers, respectively. These primers are shown in Table 5.

TABLE 5
Primers for the amplification of the truncated fragments of nucleocapsid
gene. Roman numerals I to XVI correspond to SEQ ID Nos. 10-25.

| | Size of amino acid | |
|---|---|---|
| Target gene | (Location) | Primers |
| Full length | 423aa | Forward: 5′-CGGGATCCATGTCTGATAATGGACCCCAATC-3′ (I) |
| | (1-1269 bp) | Reverse: 5′-ACGCGTCGACTTATGCCTGAGTTGAATCAGC-3′ (II) |
| N210 | 210aa | Forward: 5′-CGGGATCCATGTCTGATAATGGACCCCAATC-3′ (III) |
| | (1-630 bp) | Reverse: 5-ACGCGTCGACTCGAGCAGGAGAATTTCCCC-3′ (IV) |
| N195 | 195aa | Forward: 5′-CGGGATCCAACCAGCTTGAGAGCAAAGTTTC-3′ (V) |
| | (684-1269 bp) | Reverse: 5′-ACGCGTCGACTTATGCCTGAGTTGAATCAGC-3′ (VI) |
| N170 | 170aa | Forward: 5′-CGGGATCCGCCTTGAATACACCCAAAGAC-3′ (VII) |
| | (414-924 bp) | Reverse: 5′-ACGCGTCGACAAATTGTGCAATTTGCGGCC-3′ (VIII) |
| N71 | 71aa | Forward: 5′-CGGGATCCGCCTTGAATACACCCAAAGAC-3′ (IX) |
| | (414-627 bp) | Reverse: 5′-ACGCGTCGACAGCAGGAGAATTTCCCCT-3′ (X) |
| N80A | 80aa | Forward: 5′-CGGGATCCTTGAACCAGCTTGAGAGCAAA-3′ (XI) |
| | (684-924 bp) | Reverse: 5′-ACGCGTCGACAAATTGTGCAATTTGCGGCC-3′ (XII) |
| N80B | 80aa | Forward: 5′-CGGGATCCGATCCACAATTCAAAGACAAC-3′ (XIII) |
| | (1029-1269 bp) | Reverse: 5′-ACGCGTCGACTTATGCCTGAGTTGAATCAGC-3′ (XIV) |
| N74 | 74aa | Forward: 5′-CGGGATCCAACGTCATACTGCTGAACAAGCAC-3′ (XV) |
| | (1045-1269 bp) | Reverse: 5′-ACGCGTCGACTTATGCCTGAGTTGAATCAGC-3′ (XVI) |

Construction of Recombinant Plasmids Carrying Nucleocapsid/or Spike Protein Fragments and Transformation of Host Cells

The purified DNAs encoding N protein fragments were digested with BamHI and SalI. The purified DNAs encoding S protein fragments were digested with BamHI and SalI. The resulting fragments were cloned into pGEX or pQE expression vectors (Amersham Pharmacia) (pGEX4T-3 for N protein and G1 to G18 expression) (26) using rapid ligation kit (Roche, Germany).

The plasmid constructs were transformed into E. coli JM105, DH5 alpha and/or BL21 cells to produce GST (Glutathione S transferase) fusion proteins with a GST moiety at the carboxyl terminus. Positive clones were identified by PCR screening and further confirmed by enzyme cut and sequence analysis. The insert sequences were confirmed by corresponding N and S gene sequences.

Construction of Recombinant Baculovirus Vectors Expressing Fusion Proteins of Nucleocapsid-Spike/Spike-Nucleocapsid Fragments and Transformation of Insect Host Cells

Recombinant plasmids for the production of two fusion proteins were constructed. In one, a nucleotide acid encoding the Fc fragment (Fc gene) was cloned upstream of a nucleotide acid encoding the N195 fragment, in the other the N195 gene was cloned upstream of the Fc gene. These Fc/N195 and N195/Fc constructs were inserted into the baculovirus expression vector, pFastBac™HTa (Life Technologies, Inc.) and transfected into SF9 insect cells to obtained recombinant AcMNPV baculovirus expressing fusion protein Fc-N195 and N195-Fc, respectively. The respective virus stocks were amplified and virus titres were determined in each of the virus stocks using the viral plaque assay protocol described for the BAC-TO-BAC™ Baculovirus Expression Systems [INVITROGEN] (40). The virus titre of both virus stocks were determined to be 2×10⁷ pfu/ml.

For protein expression, SF9 insect cells were infected with a M.O.I. (multiplicities of infection) of 5 and the cells were harvested 36 h p.i. (hours post infection). Total cell lysate from cells infected with baculovirus containing the constructs described above were analyzed by western blot using rat-anti N195 and rat-anti Fc polyclonal antibodies, which had been previously produced. Proteins with the expected size of a Fc-N195 and N195-Fc fusion protein, namely 52 KDa, were successfully expressed and could be detected via Western blot.

Protein Expression and Purification

Protocol I:

A fresh overnight culture of host cells carrying various SARS virus structural gene fragments was diluted 1:25 in 1 liter LB medium containing ampicillin (100 μg/ml) and grown at 37° C. at a shaking speed of 200 rpm until OD595 reached 0.5/0.6. The culture was induced by adding isopropyl-B-D-thiogalactopyranoside to a final concentration of 0.5 mM for 4 h at 37° C. The cultures were then harvested by centrifugation at 4000×rpm for 30 min and the bacterial cell pellets were resuspended in 25 ml of lysis buffer (20 mM Tris-HC1/500 mM NaCl, 1 mM DTT pH 7.5) containing 1 mg/ml lysozyme and incubated at 4° C. for complete dissolution (Kwang et al., 1993) (27). Subsequently the cells were sonicated and the lysate was clarified by a high speed spin at 18,000 rpm for 1 h at 4° C. The supernatants were then incubated with Glutathione Sephrose4B resin (Amersham-Pharmacia) overnight at 4° C. The resin was packed into a column and washed three times with the above buffer pH (7.5). Elution of protein was accomplished with three column volumes of lysis buffer containing 20 mM reduced Glutathione (Sigma). The fraction of interest was collected and the GST tag was removed from the fusion protein by overnight thromobin treatment. After desalting, the eluate was passed through the GST column to remove the GST from the eluate. The final protein content was measured with Bio-Rad protein assay kit (Bradford, 1976) (28) and the purity was checked by Coomassie staining of the samples run on SDS-PAGE.

Protocol II:

Alternatively, the transformed bacteria were grown to an OD₆₀₀ of 0.5 to 0.6 in luria-Bertani (LB) medium with ampicillin (final concentration 100 μg/ml), and induced with 1 mM IPTG for 5 h at 37° C. Cells were pelleted and resuspended in 1×PBX. The sonicated lysate with centrifuged at 20 000×g for 10 min.

The soluble recombinant proteins were incubated with Glutathione Sepharose 4B beads (Amersham Biosciences, New Jersey) and eluted with 10 mM glutathione (Sigma, St. Louis) in 50 mM Tris-HC1, pH 8.0. The GST protein was cleaved using thrombin protease (Amersham Biosciences, New Jersey). Dialysis was performed overnight in 1×PBS at 4° C., followed by removing GST using Glutathione Sepharose 4B. However, the insoluble proteins, which were dissolved in 1 M, 6 M and 8 M urea, respectively, were purified using protein eluted (Bio-Rad, USA).

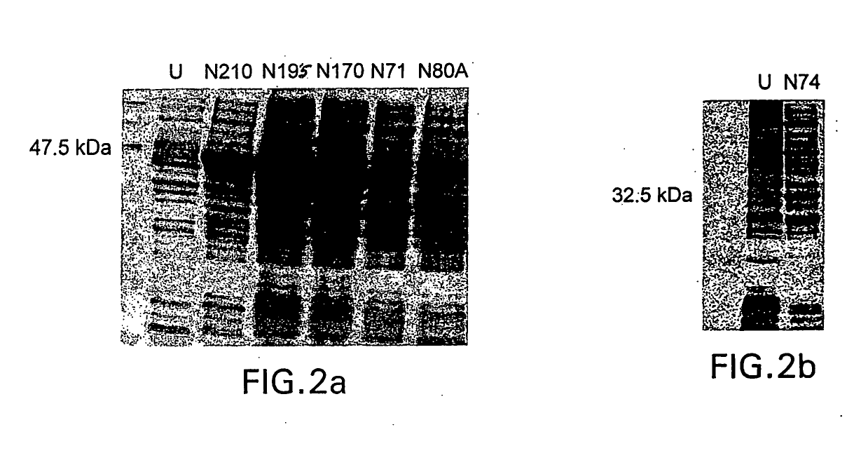

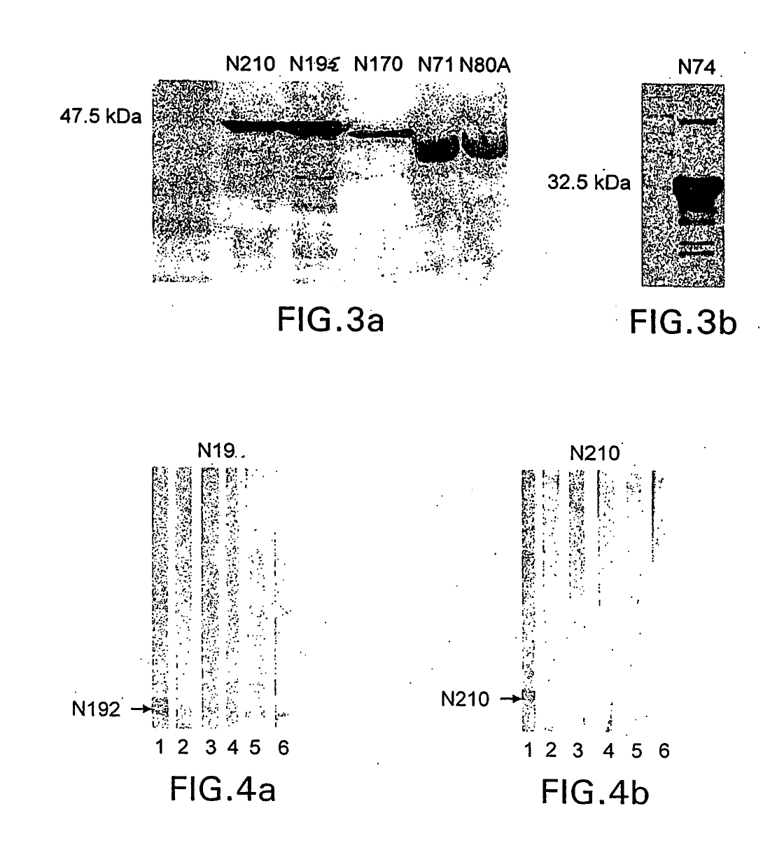

As shown in FIG. 2, expression of all N protein fragments shown therein was high.

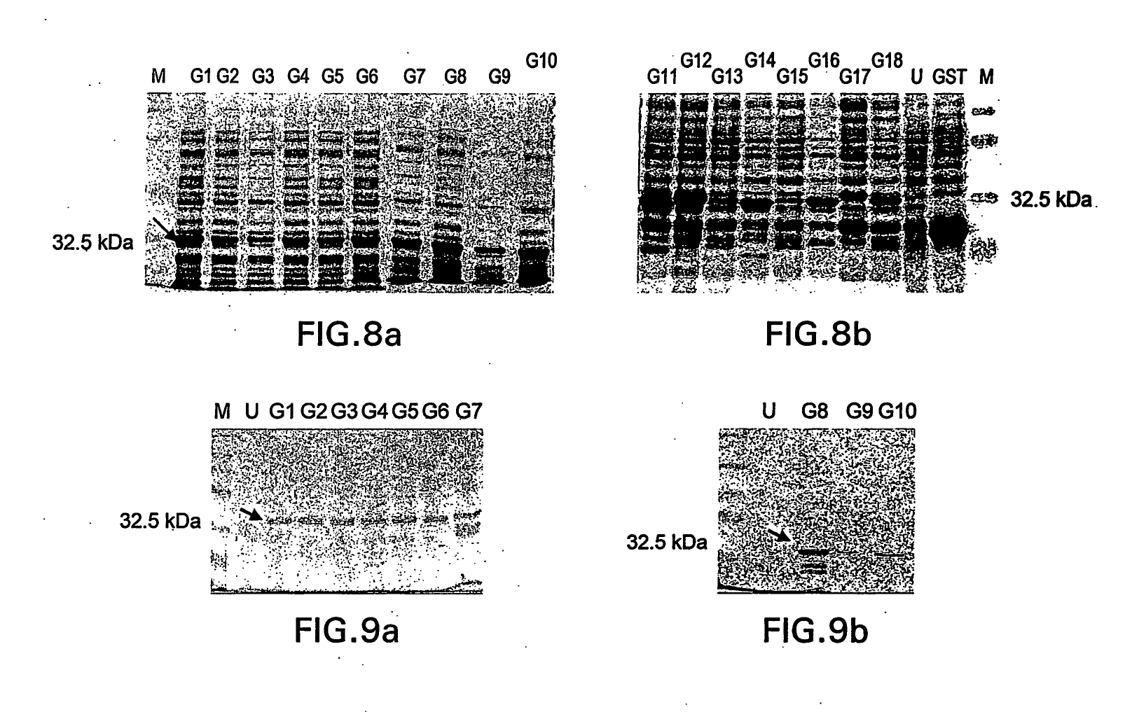

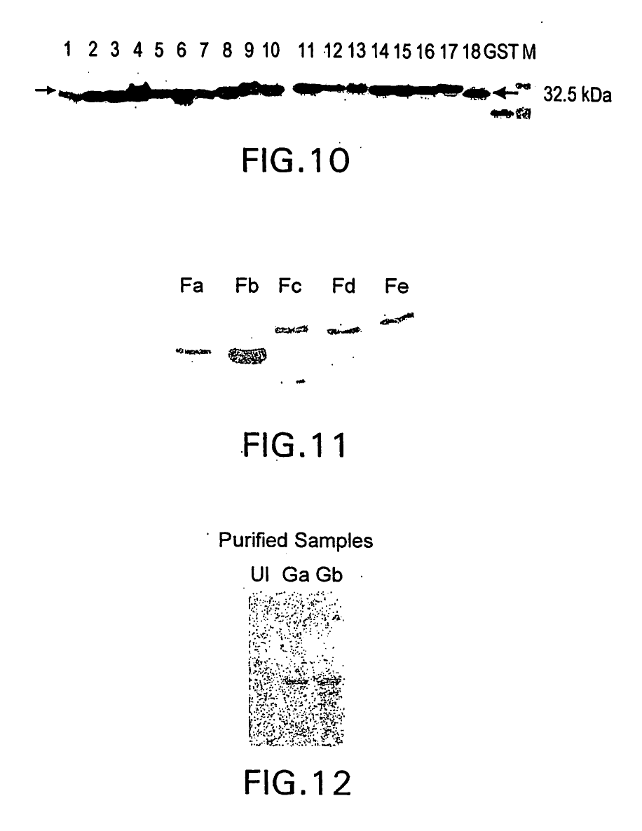

Expressed and purified S protein fragments G1-G18 are shown in FIGS. 8 and 9, respectively. Purified S protein fragments Ga and Gb are shown in FIG. 12.

Fragment N195 showed excellent protein yield and was also easy to purify.

Western Blot Protocol

Western blot assays were performed based on the standard protocols by Burnett (1981) (29) and Cabradilla et al. (1986) (30). The various purified recombinant protein fragments were separated by 12 to 15% SDS-PAGE and transferred to nitrocellulose membrane (0.45 μm) (Bio-Rad, USA) or Hybond™ nitrocellulose membranes (Bio-Rad, USA). The membranes were blocked with 5% non-fat dry milk (Bio-Rad) in PBST for 1 h at room temperature and washed with PBST once. The membranes were cut into 3 cm strips before incubating them with SARS positive and negative serum at 1:100 dilution at room temperature for 1 h. The membrane strips were then washed three times with PBST and incubated with human anti-IgG or IgM conjugated with horseradish peroxidase (HRP) (DAKO, Denmark) at room temperature for 1 h. After rinsing the strips three times with PBS, the specific reaction bands were visualized by DAB (3,3′-diaminobenzidine tetrahydrochloride; Pierce, Ill., USA; HRP substrate) incubation for 3-5 min at room temperature.

ELISA

The ELISA assays were performed based on the protocol of Kwang et al. (1993) (26). The purified recombinant protein 75 ng/n 100 μl of bicarbonate/carbonate coating buffer pH (9.6) was coated on 96-well microtiter plates (CovaLink plates, Nunc, Denmark). The plate was then left at 4° C. overnight, and the wells were blocked subsequently with blocking buffer (5% W/C non-fat dry milk 0.2% Tween 20, 0.02% sodium azide in PBS) for 10 min at 37° C. to saturate the excess binding sites. The wells were washed three times with PBS-tween-20 and 100 μl per well of human SARS positive and negative serum diluted in 1% blocking buffer was added and left at 37° C. for 10 min. The plate was then washed three times before adding 100 μl per well of secondary antibody (anti-human immunoglobulin G (IgG)—conjugated with horseradish peroxidase (HRP) DAKO, Denmark) diluted in PBST and incubated at 37° C. for 10 min. After further washing, 50 μl of O-phenylenediamine dihydrochloride color-development reagent (Sigma) were added to each well and incubated for 5 min at room temperature. The reaction was stopped by adding 12.5 μl of 4 N sulfuric acid and the plate was read at 492 nm.

Immunofluorescence Assay (IFA)

The Immunofluorescence assay was performed in laminar-flow safety cabinets in a biosafety level 3 (BSL-3) laboratory. SARS coronavirus was propagated in Vero E6 cells at 37° C. until cytopathogenic effects were seen in 75% of the cell monolayer, following which the cells were harvested, spotted onto Teflon coated slides and fixed with 80% cold acetone. Serum samples were tested at 1:10 dilution and washed with 1×PBS after being incubated either for 90 min, followed by fluorescein isothiocyanate (FITC)-conjugated rabbit anti-human immunoglobulin M (IgM) or for 30 min, followed by FITC-conjugated anti-human immunoglobulin G (IgG) and incubated for a further 37° C. The slides were subjected to another washing cycle before being read for specific fluorescence under an immunofluorescence microscope.

Immunofluorescence Assay (IFA) Using Protein Fragments

SF9 insect cells were cultured in 96 well plate with 60% confluency. Two sets of SF9 cells were infected with baculoviruses expressing fusion protein Fc-N195 and N195-Fc with a M.O.I. of 5. The cells were fixed with 100% ethanol for 30 minutes at 36 h p.i. To optimize the IFA procedure, the fixed SF9 cells were tested with varying dilutions of infected patient serum as primary antibody and FITC-conjugated rabbit anti-human IgG or IgM as secondary antibody for each IgG and IgM detection. The best concentration of primary antibody to be used for IgG and IgM IFA detection was determined as 1:100 and 1:10, respectively, based on the fluorescence signals and reaction background.

86 sera with 21 from confirmed SARS infected patients (Table 6) were tested. The results were compared with those obtained with a western blot assay, whole virus IFA test and commercially available IFA kit (EUROIMMUN AG), which also uses inactivated whole SARS virus as antigen. As can be seen from Table 6, the IFA of both fusion proteins (Fc-N195 and N195-Fc) showed comparable results in term of sensitivity and specificity to the commercial kit and whole virus IFA. The modified IFA using the two fusion proteins showed a better detection rate than Western Blot analysis.

TABLE 6

| | | 2s-59 | 2s-73 | 3s-17 | 3s-20 | 3s-24 | 3s-42 | 4-7 | 5-4 | 5-12 | 5-20 | 5-28 |
|---|---|---|---|---|---|---|---|---|---|---|---|---|
| FC-N195¹ | IgG | +++ | ++++ | +++ | +++ | ++ | ++++ | ++++ | +++ | ++ | − | +++ |
| | IgM | − | ++ | +++ | + | − | + | − | + | + | + | − |
| N195-Fc² | IgG | + | ++ | ++++ | +++ | ++ | +++ | ++ | ++ | + | − | +++ |
| | IgM | + | − | + | + | − | ++ | + | + | + | + | + |
| Commercial³ | IgG | ++ | ++ | ++ | ++ | + | + | + | + | + | − | ++ |
| | IgM | + | − | ++ | + | − | − | +++ | − | + | + | + |
| Whole Virus⁴ | IgG | NT | NT | +++ | +++ | +++ | +++ | NT | − | NT | + | − |
| | IgM | NT | NT | + | + | + | ++ | NT | + | NT | − | + |
| Western Blot⁵ | IgG | +++ | ++ | + | ++++ | ++++ | + | + | +++ | ++ | − | ++++ |
| | IgM | − | − | ++ | ++ | − | +++ | − | +++ | +++ | − | − |

| | | 8-1 | 8-2 | 8-3 | 8-4 | 8-5 | 8-6 | 8-7 | 8-8 | 8-9 | 8-10 |
|---|---|---|---|---|---|---|---|---|---|---|---|
| ScN195¹ | IgG | ++ | + | ++ | +++ | ++ | +++ | +++ | ++++ | ++++ | +++ |
| | IgM | + | − | + | + | + | − | ++ | − | + | +++ |
| N195Sc² | IgG | ++++ | ++ | ++ | +++ | + | ++ | +++ | ++ | ++ | +++ |
| | IgM | + | + | ++ | + | + | − | + | − | − | + |
| Commercial³ | IgG | +++ | ++++ | +++ | ++ | +++ | ++ | ++ | ++ | ++ | ++ |
| | IgM | + | ++ | +++ | + | + | + | − | ++ | + | + |
| Whole Virus⁴ | IgG | + | + | + | + | + | + | + | + | + | + |
| | IgM | + | + | + | + | + | + | + | + | + | + |
| Western Blot⁵ | IgG | +++ | + | + | +++ | ++ | ++ | ++ | ++++ | ++++ | +++ |
| | IgM | + | − | − | + | + | − | + | − | − | ++ |

¹Recombinant baculovirus expressed Fc-N195 fusion protein
²Recombinant baculovirus expressed N195-Fc fusion protein
³Commercially available IFA test using whole SARS virus as antigen (EUROIMMUN AG)
⁴Whole virus IFA test from hospital based in Singapore
⁵Recombinant N195 based western blot assay

Production of Monoclonal Antibodies Against S and N Protein

Fragments Fc and N195 were expressed and purified as described above, mixed with montanide adjuvant (SEPPIC) and injected into mice. After booster shots at intervals of two weeks, spleen-cells were extracted and fused with myeloma cells to form hybridoma cells to produce specific monoclonal antibody against N protein and S protein, respectively. Cells fusion was performed essentially as described by Yokoyama (39). Briefly, SP2/0 myeloma cells were fused with spleen cells using 50% polyethyleneglycol. Cells were plated at a density of 105 cells/well in well tissue culture plates. Individual wells were examined for growth and the supernatants of wells with growth were screened for S and N specific antibodies by ELISA using purified S and N target protein, respectively. Cells with the desired specificity were expanded and hybridoma cells with high growth rate were grown in 75 cm² flasks at 37° C. incubation for mass production of monoclonal antibody.

Determination of Cross Reactivity of N Protein Fragments with Sera Against Non SARS Coronaviruses

The reactivity of the N protein fragments with chicken serum against avian Infectious Bronchitis Virus (IBV) and pig serum against transmissible gastroenteritis (TGE) was tested using western blot assays. Substantial cross-reactivity was observed. It was hypothesized that this might be an effect of the GST moiety at the amino terminus of the fusion protein.

Accordingly, the GST moieties were cleaved from the fusion proteins by thrombin protease to release the N protein fragments.

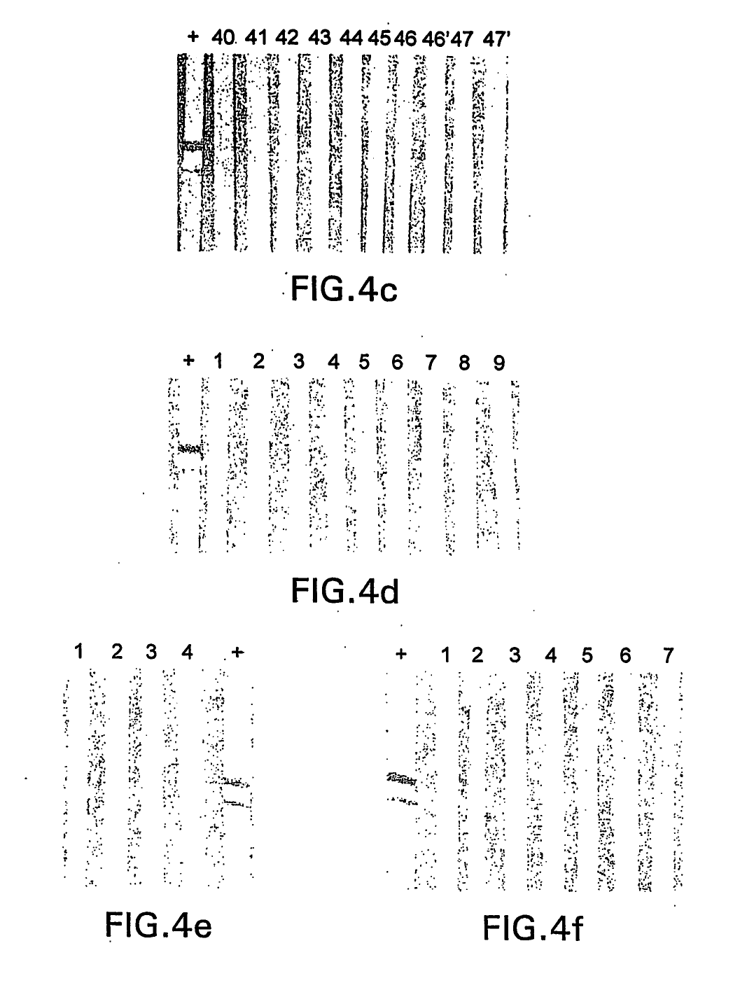

The released N protein fragments were again tested with chicken serum against avian Infectious bronchitis virus (IBV) and pig serum against transmissible gastroenteritis (TGE). As shown in FIGS. 4(a) and 4(b), lanes 3-6, in particular lanes 3 and 5, N protein 195 and N protein 210 did not show cross reactivity with either of the sera, nor did any of other fragments tested.

N195 was tested for reactivity with sera from (I) cats infected with cat coronavirus, (ii) dogs infected with dog coronavirus, (iii) chicken infected with avian coronavirus, (iv) pig infected with porcine coronavirus. As can be seen from FIGS. 4 (d) to (f), no cross reactivity was observed.

Determination of Cross Reactivity of S Protein Fragments with Sera Against Non SARS Coronaviruses

The reactivity of isolated and purified protein fragments Fa-Fe were tested with chicken serum against avian Infectious Bronchitis Virus (IBV) and pig serum against transmissible gastroenteritis (TGE). Fragments Fa to Fe did not show cross reactivity with either of the sera.

Determination of Reactivity of N Protein Fragments with SARS Positive and SARS Negative Sera

All N protein fragments were tested with sera of infected and uninfected humans.

Fragments N170, N71, N80 and N74 only reacted with some of the tested sera from patients infected with the SARS virus. Fragments N210 and N195 were found to be immunodominant.

Both fragment N210 and fragment N195 were reacted with 33 SARS positive sera and did not react with 66 SARS negative sera. As can be seen from Table 6, the N195 IgM detection rate was, however, substantially higher than that of N210. The results shown in Table 7 were obtained by western blot analysis.

TABLE 7
Detection patterns of the N210 and N195 proteins

| | Sera Descriptions | N210 | N195 |
|---|---|---|---|
| IgG detection | SARS positive (33 samples) | 33/33 | 33/33 |
| | SARS negative (66 samples) | 0/66 | 0/66 |
| IgM detection | SARS positive (33 samples) | 3/33 | 15/33 |
| | SARS negative (66 sample) | 0/66 | 0/66 |

Determination of Reactivity of S Protein Fragments with SARS Positive Sera

All S protein fragments were tested with infected human serum samples and showed positive reactions. As can be seen from Table 9, Fc includes an immunodominant dominant of the spike protein and reacted with all 10 SARS patient serum samples tested.

TABLE 8
Reactivity of the 18 GST-fusion S protein fragments against 10 convalescent SARS positive serum samples

| | Epitope no. | | | | | | | | | | | | | | | | | |
|---|---|---|---|---|---|---|---|---|---|---|---|---|---|---|---|---|---|---|
| Serum no. | G1 | G2 | G3 | G4 | G5 | G6 | G7 | G8 | G9 | G10 | G11 | G12 | G13 | G14 | G15 | G16 | G17 | G18 |
| 1 | − | − | − | − | − | + | + | + | + | + | + | − | − | + | − | − | + | − |
| 2 | − | − | − | − | − | − | − | − | − | − | − | − | − | − | − | − | − | − |
| 3 | − | − | − | − | − | − | + | + | + | + | + | + | + | + | − | − | − | − |
| 4 | − | − | − | − | − | − | − | − | + | − | − | − | − | − | − | − | − | − |
| 5 | − | − | − | − | − | − | − | − | − | − | − | − | − | − | − | − | − | − |
| 6 | − | − | − | − | − | + | + | − | + | − | − | − | − | − | − | − | − | − |
| 7 | − | − | − | − | − | − | − | − | − | − | − | − | − | − | − | − | − | − |
| 8 | − | − | − | − | − | − | − | − | + | − | − | − | − | − | − | − | − | − |
| 9 | − | − | − | − | − | − | − | − | + | − | − | − | − | − | − | − | − | − |
| 10 | − | − | − | − | − | − | − | − | + | − | − | − | − | − | − | − | − | − |
| Total no. of | − | − | − | − | − | 2 | 3 | 3 | 6 | 2 | 2 | 1 | 1 | 2 | − | − | 1 | − |

reactive sera

TABLE 9
Reactivities of 10 SARS patient serum samples with fragments
Fa-Fe of S protein expressed from insect cells.

| | Serum | | | | | | | | | | | |
|---|---|---|---|---|---|---|---|---|---|---|---|---|
| | | | | | | | | | | | | Number |
| | | | | | | | | | | | | of |
| Do- | Normal | | | | | | | | | | | reactive |
| main | Serum | 1 | 2 | 3 | 4 | 5 | 6 | 7 | 8 | 9 | 10 | sera |
| Fa | − | − | − | − | − | − | − | − | − | − | − | 0 |
| Fb | − | − | − | − | − | − | − | − | − | − | − | 0 |
| Fc | − | + | + | + | + | + | + | + | + | + | + | 10 |
| Fd | − | + | − | + | + | − | + | − | + | − | + | 6 |
| Fe | − | + | − | − | − | − | − | − | − | − | − | 1 |

Inoculation of Mice and Guinea Pig with S Protein Fragments

Fragments Fa to Fe were expressed in a baculovirus system. Mice and guinea pigs were inoculated with these fragments two times.

Clinical Tests

Western Blot Assay Using N195

A clinical sample comprising 274 sera was used in a blinded test to test the accuracy and repeatability of a SARS infection with a western blot using N195. The clinical sample also included multiple tested and patient time course samples. From the blinded test, 40 samples tested positive. The detection rate was 88.6% (39/44) for IgG antibodies and 56.8% (25/44) for IgM antibodies, respectively. Combination of these two numbers gave a overall detection rate of 90.9%. The 40 positive testing samples matched the respective hospital records (44 SARS confirmed cases). The results are illustrated in Table 10. The Table shows that the western blot test results were highly concordant with the clinical diagnosis. It can be seen that from 100 samples from patients suffering from autoimmune diseases (SLE, connective tissue diseases and inflammatory arthritis), only four showed non-specific reaction in the western blot.

TABLE 10

| | | | | Specificity/ |
|---|---|---|---|---|
| Serum | | Patient | | sensitivity |
| group | number | Sera description | Result | rate |
| Clinically | 274 | Samples from | 40 | 90.9% |
| blinded | | a) SARS patients | positive | sensitivity |
| samples | | (4-76 days post fever) | out of 44 | and 98.3% |
| | | b) Autoimmune disease | SARS | specificity |
| | | patients* | confirmed | |
| | | c) Dengue patients | patients | |
| | | d) Aspiration and | | |
| | | community acquired | | |
| | | pneumonia patients | | |
| | | e) Renal failure patients | | |
| | | f) Other diseases patients | | |

*4 out of 100 autoimmune diseases showed non-specific reaction in a
N195 based western blot
$\begin{matrix}Sensitivity = \frac{True positive samples}{True positive samples + False negative samples} \% \\ = [ 40 / ( 40 + 4 ) ] \% \\ = 90.9 \%\end{matrix}$
$\begin{matrix}Specificity = \frac{True negative samples}{True negative samples + False positive samples} \% \\ = [ 226 / ( 226 + 4 ) ] \% \\ = 98.3 \%\end{matrix}$

Among the 40 SARS positive samples collected between 4 to 76 after fever onset, the detection rate for IgG antibodies was higher than for IgM. This is believed to be a consequence of the fact that on average the sera were collected relatively late with respect to fever and cough onset. The western blot employed could detect IgG at a dilution of about 1:800 and IgM at a dilution of about 1:100.

Table 11 shows the specific results obtained for 39 patients tested. As shown in the table some of the patients listed had clinical SARS status, while others had not. The table also shows three samples selected from the same patient at different time points (patient No. 15, 16 and 17). For this patient, SARS antibody detection was negative at 7 days post onset but was positive at 15 and 23 days post onset. These samples also confirmed repeatability of the assay. The table also shows samples from patients that had fever symptoms at the time tested, but otherwise did not met the criteria for SARS at the time when SARS was epidemic in Singapore. All of these samples tested negative for SARS coronavirus IgM and IgG antibodies using the western blot.

Table 11 also compares the results obtained for the listed patients to results obtained via IFA, that is based on whole SARS virus. The shown samples tested with IFA included 20 western blot SARS positive samples, 5 western blot negative by suspected samples (4-17 days post fever) and 14 samples from other diseases. Both IFA and western blot showed 20 positive and 10 negative samples. Patient nos. 18 and 20 showed non-specific reactions by western blot, while patient no. 24, 25, 26 and 27 showed positive or non-specific results in the IFA test only. Samples of patient nos. 34 and 35 showed non-specific results using either method. Accordingly, the overall detection rate, specificity and selectivity obtained using N195 in a western blot compared well with the overall detection rate, specificity and selectivity obtained via IFA.

TABLE 11
Comparison of western blot and IFA of 39 selected samples

| | | Clini- | | | | | |
|---|---|---|---|---|---|---|---|
| Pa- | | cally | | Western blot | | IFA | |
| tient | Patient | SARS | Days of | detection | | detection | |
| No. | record | status | fever | IgG | IgM | IgG | IgM |
| 1. | 1-SS4 | + | unknown | ++++* | −* | +++ | − |
| 2. | 1-SS10^{§} | − | − | − | − | − | − |
| 3. | 1-SS13^{§} | − | − | − | − | − | − |
| 4. | 1-SS16^{§} | − | − | − | − | − | − |
| 5. | 1-SS18^{§} | − | − | − | − | − | − |
| 6. | 1-SS19^{§} | − | − | − | − | − | − |
| 7. | 2-SS46^{§} | − | − | − | − | − | − |
| 8. | 2-SS59 | + | 26 | +++ | − | +++ | + |
| 9. | 2-71 | + | 8 | − | + | − | + |
| 10. | 3-S10 | + | 4 | − | − | − | − |
| 11. | 3-S17 | + | 4 | + | ++ | +++ | + |
| 12. | 3-S24 | + | 74 | ++++ | − | +++ | + |
| 13. | 3-S20 | + | 49 | ++++ | ++ | +++ | + |
| 14. | 3-S38 | + | 76 | ++ | ++++ | − | − |
| 15. | 3-S40^{†} | + | 7 | − | − | − | − |
| 16. | 3-S41^{†} | + | 15 | + | ++ | +++ | ++ |
| 17. | 3-S42^{†} | + | 23 | + | +++ | +++ | ++ |
| 18. | 5-1^{‡} | − | − | NSR [symbol] | − | − | − |
| 19. | 5-4 | + | unknown | +++ | ++ | − | + |
| 20. | 5-25^{‡} | − | − | NSR | − | − | NSF^{¶} |
| 21. | 5-28 | + | unknown | − | ++++ | − | + |
| 22. | 5-32 | + | 12 | − | − | − | − |
| 23. | 7-7 | + | 17 | − | − | + | − |
| 24. | 6-2^{‡} | − | − | − | − | − | + |
| 25. | 6-3^{‡} | − | − | − | − | − | NSF |
| 26. | 6-4^{‡} | − | − | − | − | − | Weak |
| | | | | | | | posi- |
| | | | | | | | tive |
| 27. | 6-5^{‡} | − | − | − | − | − | NSF |
| 28. | 7-11 | + | 14 | + | − | + | − |
| 29. | 7-12 | + | 13 | +++ | + | + | − |
| 30. | 7-13 | + | 13 | ++++ | +++ | ++ | − |
| 31. | 7-15 | + | 7 | − | − | − | − |
| 32. | 7-16 | + | unknown | + | + | − | + |
| 33. | 7-17 | + | 13 | +++ | + | ++ | − |
| 34. | 7-21^{‡} | − | − | NSR | NSR | NSF | NSF |
| 35. | 7-24^{‡} | − | − | NSR | NSR | NSF | NSF |
| 36. | 9-1 | + | unknown | + | + | +++ | + |
| 37. | 4299 | + | 11 | ++ | +++ | − | + |
| 38. | 2604:4209 | + | 11 | +++ | + | +++ | + |
| 39. | 1605:4153 | + | 31 | +++ | − | +++ | − |

| | Western blot | IFA |
|---|---|---|
| Overall detection of SARS coronavirus: | 20/25 | 20/25 |

*Number of plus indicated the degree of positive signals, while minus denoted negative result or negative signals.
^{†}Patient no. 15, 16 and 17 were consecutively collected from one patient.
^{‡}Autoimmune diseases.
^{§}Other diseases.
NSR [symbol] /NSR: Non-specific reaction.
NSF^{¶}/NSF: Non-specific fluorescence.

### REFERENCES

(1) Wenzel R P and Edmond M B, Managing SARS amidst Uncertaincy, N. Eng. J. Med., Vol. 348, No. 20, p. 1947-1948 (May 15, 2003).
(2) Nie Q H, Luo X D, Hui W L, Advances in clinical diagnosis and treatment of severe acute respiratory syndrome. World J. Gestroenterol. (2003); 9:1139-43.
(3) Peiris J S, Lai S T, Poon L L, Coronavirus as a possible cause of severe acute respiratory syndrome. Lancet. (2003); 361: 1319-25.
(4) Ksiazek T G, Erdman D, Goldsmith C S. A novel coronavirus associated with severe acute respiratory syndrome. N Engl. J. Med. (2003); 348: 1953-66.
(5) Drosten C, Gunther S, Preiser W. Identification of a novel coronavirus in patients with severe acute respiratory syndrome. N Engl. J. Med. (2003); 348; 1967-76.
(6) Poutanen S M, Low D E, Henry B. Identification of severe acute respiratory syndrome in Canada. N Engl J. Med. (2003); 348: 1995-2005.
(7) Rota P A, Obserste M S, Monroe S S, Nix W A, Campagnoli R. Icenogle J P, et al. Characterization of a novel coronavirus associated with severe acute respiratory syndrome. Science. (2003); 300: 1394-9.
(8) Marra M A, Jones S J, Astell C R. The Genome sequence of the SARS-associated coronavirus. Science. (2003); 300:1399-404.
(9) Alan J. Cann, Principles of Molecular Virology: Genomes, p. 80-81 (3^{rd} ed., Academic Press, 2001)
(10) Zwaagstra K A, van der Zeijst B A, Kusters J G. Rapid detection and identification of avian infectious bronchitis virus. J Clin Microbiol. (1992); 30: 79-84.
(11) Kubota S, Sasaki O, Amimoto K, Okada N, Kitazima T, Yasuhara H. Detection of porcine epidemic diarrhea virus using polymerase chain reaction and comparison of the nucleocapsid protein genes among strains of the virus. J Vet Med Sci. (1999); 61: 827-30.
(12) Falcone, E, D'Amore, E, Di Trani L, et al. Rapid diagnosis of avian infectious bronchitis virus by the polymerase chain reaction. J Virol Methods. (1997); 64:1235-30.
(13) Annu Alho, Jane Marttila, Jorma Ilonen, Timo Hyypia. Diagnostic potential of Parechovirus capsid protein. J Clin Microbiol. (2003); 41: 2294-2299.
(14) Stohlman S A, Bergmann C, Cua D, Wege H, van der Veen R. Location of antibody epitopes within the mouse hepatitis virus nucleocapsid protein. Virology. (1994); 202:146-53.
(15) Seah J N, Yu L, Kwang J, Localization of linear B-cell epitopes on infectious bronchitis virus nuceocapsid protein. Vet. Microbiol. (2000); 75:11-6.
(16) Kathryn V. Holmes, SARS-Associated Coronavirus, N. E. J. Med., Vol. 348, No. 20, p. 1948-1951 (May 15, 2003).
(17) Thomas G. Ksiazek et al., A Novel Coronaviruse Associated with Severe Acute Respiratory Syndrome, N. Eng. J. Med., Vol. 348, No. 20, p. 1953-1966 (May 15, 2003).
(18) World Health Organization, Use of Laboratory Methods for SARS Diagnosis, http://www.who.int/csr/sars/labmethods/en/ as of Oct. 2, 2003.
(19) Kamps & Hoffman, Sars Reference 7-2003, Chapter 7: Diagnostic Tests (Flying Publisher, 2^{nd} ed., Jul. 10, 2003) http://www.sarsreference.com as of Oct. 2, 2003.
(20) Ruan Y J, Wei C L, Ling A E, Vega V B, Thoreau H, Su S T, et al. Comparative full-length genome sequence analysis of 14 SARS coronavirus isolates and common mutations associated with putative origins of infection. Lancet (2003); 361: 1779-85.
(21) Peiris, J S M, Chu, C M, Cheng, V C C, et al. Clinical progression and viral load in a community outbreak of coronavirus-associated SARS pneumonia: a prospective study. Lancet. (2003); 361: 1767-72.
(22) US Center for disease Control and Prevention (CDC). Updated interim U.S. case definition of severe acute respiratory syndrome (SARS). Atlanta: The CDC; 2003 Jul. 18. Available: http://www.cdc.gov/ncidod/sars/casedefinition.htm (Accessed 2003 Oct. 2, 2003).
(23) Martin J. Bishop, ed., Academic Press, San Diego, 1994, and Carillo, H., and Lipman, D., SIAM J Applied Math. 48: 1073 (1988).
(24) 2 can, Bioinformatics Educational Resource; http://www.ebi.ac.uk/2can/disease/SARS.html as of Oct. 2, 2003.
(25) The nucleotide sequence of SIN2774 (Accession No. AY283798) is accessible via e.g. http://www.ncbi.nlm.nih.gov/entrez/query.fcgi (Nucleotide); Ruan, Y., Wei, C. L. et al., Comparative whole genome sequence analysis of 9 SARS coronavirus isolates show mutations in functional domains and potential geographical variations (submitted to Apr. 27, 2003 to the EMBL/GenBank/DDBL databases).
(26) Sambrook, J., Fritsch, E. F. and Maniatis, T. (2001) Molecular Cloning: A Laboratory Manual, 3^{rd} edition.
(27) Kwang, J. Keen, J. Cutlip, R. C. and Littedike, E. T., Evaluation of an ELISA for the detection of ovine progressive pneumonia antibodies using a recombinant transmembrane envelope protein. J. Vet. Diagn. Invest. (1993), 5:189-193.
(28) Bradford, M M, A rapid and sensitive method for the quantification of micro quantities of protein utilizing the principle of protein-dy binding. Anal Biochem. (1976), 72:248-254
(29) Burnett, W. N., Western blotting. Electrophoretic transfer of protein from SDS-polyacrylamide gels to unmodified nitrocellulose and radiographic detection with antibody and radioiodinated protein A. Anal. Biochem. (1981), 112:195-203
(30) Cabradilla, C. D., Groopman, J. E., Lanigan, J. et al. (1986) Serodiagnosis of antibodies to the human AIDS retrovirus with a bacterially synthesized env polypeptide. Bio/Technology 4:128-133.
(31-35) Computational Molecular Biology, Lesk, A. M., ed., Oxford University Press, New York, 1988.
Biocomputing: Informatics and Genome Projects, Smith, D. W., ed., Academic Press, New York, 1993.
Computer Analysis of Sequence Data, Part I, Griffin, A. M., and Griffin, H. G., eds., Humana Press, New Jersey, 1994.
Sequence Analysis in Molecular Biology, von Heinje, G., Academic Press, 1987.
Sequence Analysis Primer, Gribskov, M. and Devereux, J., eds., M Stockton Press, New York, 1991.
(36) Devereux, J., et al., A Comprehensive Set of Sequence Analysis Programs for the VAX. Nucleic Acids Research 12(1): 387 (1984).
(37-38) Altschul et al., Basic local alignment search tool. J. Mol. Biol. (1990) 215:403.
Altschul et al., Gapped BLAST and PSI-BLAST: a new generation of protein database search programs. Nucl. Acids Res. (1997), 25:3389-3402.
(39) Wayne M. Yokoyma, in: Current Protocols in Cell Biology, pp. 16.1.1-16.1.17., eds. J. S. Bonifacino, M. Dasso, J. B. Harford, J. Lippincoft-Schwartz, and K. M. Yamada, 1999.
(40) INVITROGEN, BAC-TO-BAC™ Baculovirus Expression Systems, Instruction Manual, 5.13 Viral Plaque Assay (2002).

1. A diagnostic method for detecting in at least one biological sample an antibody that binds to at least one epitope of a SARS virus, comprising:
(a) contacting said at least one biological sample with
at least one isolated SARS virus protein, or
at least one fragment of said isolated SARS virus protein comprising at least one epitope of the SARS virus, and
(b) detecting the formation of an antigen-antibody complex between said virus protein or said fragment and an antibody present in said biological sample.

2. The method of claim 1, wherein said at least one isolated SARS virus protein is an N or S protein.

3. The method of claim 2, wherein said at least one fragment
(a) is N195 or Fc of SIN 2774;
(b) corresponds substantially to N195 or Fc of SIN 2774;
or a mixture thereof.

4. The method of claim 1, wherein said at least one isolated SARS virus protein or fragment thereof is a recombinant expression product.

5. An in vitro diagnostic kit for detecting in a biological sample an antibody against a SARS virus comprising:
(a) at least one isolated SARS virus protein, or
at least one fragment of said isolated SARS virus protein comprising at least one epitope of the SARS virus, and
(b) reagents for detecting the formation of antigen-antibody complex between said at least one isolated SARS virus protein or a fragment thereof and at least one antibody present in said biological sample,
wherein said at least one isolated protein or fragment thereof and said reagents are present in an amount sufficient to detect the formation of said antigen-antibody complex.

6. The kit of claim 5, wherein said at least one fragment
(a) is N195 or Fc of SIN 2774;
(b) corresponds substantially to N195 or Fc of SIN 2774;
or a mixture thereof.

7. A method for determining an epitope specific for the SARS virus comprising:
(a) providing at least one fragment of at least one protein of the SARS virus, wherein said at least one fragment is at least 65 amino acids long,
(b) reacting said at least one fragment with
(1) at least one serum sample from at least one SARS positive human, and
(2) at least one serum sample from a coronavirus positive, SARS negative, human or non-human animal,
(c) detecting fragment-antibody complexes formed from the reactions of (b) (1) and (b) (2); and
(d) selecting one or more fragments comprising epitopes specific for the SARS virus by selecting fragments that form fragment-antibody complexes as a result of the reaction of step (b) (1), but not as a result of the reaction of step (b) (2).

8. The method of claim 7, wherein said fragment is reacted with sera from at least 5 SARS positive humans.

9. The method of claim 7, wherein said serum sample in (b)(2) is chicken serum against IBV or pig serum against TGE.

10. A diagnostic method for detecting the presence in at least one biological sample of at least one antibody against a SARS virus, comprising:
(a) contacting said at least one biological sample with
one or more peptides comprising at least about 65 contiguous amino acid residues of SEQ ID No. 2, or
one or more peptides comprising at least about 65 amino acid residues and having at least about 90% sequence identity with a contiguous number of amino acid residues of SEQ ID No. 2 having about equal length as said one or more peptides, wherein said one or more peptides comprise at least one epitope of a SARS virus, and
(b) detecting whether an antigen-antibody complex has formed between said one or more peptides and antibodies present in said biological sample.

11. A diagnostic method for detecting the presence in at least one biological sample of an antibody against a SARS virus, comprising:
(a) contacting said at least one biological sample with
one or more peptides comprising at least about 65 contiguous amino acid residues of SEQ ID No. 4, or
one or more peptides comprising at least about 65 amino acid residues and having at least about 90% sequence identity with a contiguous number of amino acid residues of SEQ ID No. 4 having about equal length as said one or more peptides, wherein said one or more peptides comprise at least one epitope of a SARS virus, and
(b) detecting whether an antigen-antibody complex has formed between said one or more peptides and antibodies present in said biological sample.

12. The diagnostic method of claim 10, wherein said one or more peptides have at least about 95% sequence identity with a contiguous number of amino acid residues of SEQ ID No. 6 having about equal length as said one or more peptides.

13. The diagnostic method of claim 11, wherein said one or more peptides have at least about 95% sequence identity with a contiguous number of amino acid residues of SEQ ID No. 8 having about equal length as said one or more peptides.

14. An isolated and purified nucleic acid comprising
at least one polynucleotide comprising at least about 195 contiguous nucleotides of SEQ ID No. 1, or
at least one polynucleotide comprising at least about 195 contiguous nucleotides which have at least about 75% homology with a contiguous number of nucleotides of SEQ ID No. 1 having about equal length as said at least one polynucleotide, wherein said polynucleotide encodes a peptide that is adapted to detect anti-SARS antibody in a sample.

15. An isolated and purified nucleic acid comprising
at least one polynucleotide comprising at least about 195 contiguous nucleotides of SEQ ID No. 3, or
at least one polynucleotide comprising at least about 195 contiguous nucleotides which have at least about 75% homology with a contiguous number of nucleotides of SEQ ID No. 3 having about equal length as said at least one polynucleotide, wherein said polynucleotide encodes a peptide that is adapted to detect anti-SARS antibody in a sample.

16. An isolated and purified nucleic acid according to claim 14, wherein said polynucleotide hybridizes under stringent conditions with a contiguous number of nucleotides of SEQ ID No. 5 having about equal length as said at least one polynucleotide.

17. An isolated and purified nucleic acid according to claim 15, wherein said polynucleotide hybridizes under stringent conditions with a contiguous number of nucleotides of SEQ ID No. 7 having about equal length as said at least one polynucleotide.

18. The diagnostic method of claims 1, 10 or 11, wherein the formation of antigen-antibody complex is detected by radioimmunoassay (RIA), enzyme linked immunosorbent assay (ELISA), immunofluorescence assay (IFA), dot blot or western blot.

19. The diagnostic method of claims 1, 10 or 11, wherein the formation of antigen-antibody complex is detected by western blot and said at least one fragment or peptide is adapted to detect IgG at a dilution of about 1:800.

20. The diagnostic method of claims 1, 10 or 11, wherein the formation of antigen-antibody complex is detected by western blot and said at least one fragment or peptide is adapted to detect IgM at a dilution of about 1:100.

21. The diagnostic method of claims 1, 10 or 11, wherein the formation of antigen-antibody complex is detected by western blot and said at least one fragment or peptide has a sensitivity of more than about 85%.

22. The diagnostic method of claims 1, 10 or 11, wherein the formation of antigen-antibody complex is detected by western blot and said at least one fragment or peptide has a specificity of more than about 85%.

23. The diagnostic method of claims 1, 10 or 11, wherein the formation of antigen-antibody complex is detected by western blot and said at least one fragment or peptide has an overall detection rate for a clinical sample of more than 65%.

24. The diagnostic method of claims 1, 10 or 11, wherein said biological sample is contacted with at least two fragments of said at least one isolated SARS protein.

25. The diagnostic method of claim 24, wherein said at least two fragments are derived from at least two distinct isolated SARS proteins.

26. The diagnostic method of claim 24, wherein said at least two fragments form a fusion protein.

27. The diagnostic method of claim 24, wherein said at least two fragments are Fc and N195.

28. The diagnostic method of claim 26, wherein said fusion protein comprises Fc at its N terminus and N195 at its C terminus.

29. The diagnostic method of claim 26, wherein said fusion protein comprises N195 at its N terminus and Fc at its C terminus.

30. A method for producing a monoclonal antibody against at least one SARS protein comprising:
(a) injecting at least one antigenic fragment of said protein into a non-human animal,
(b) isolating at least one spleen cell from said non-human animal,
(c) fusing said at least one spleen cell with a myeloma cell,
(d) screening the resulting hybridoma cells with said at least one SARS protein for the production of monoclonal antibody against said at least one SARS protein, and
(e) selecting at least one hybridoma cell producing said monoclonal antibody.

31. The method of claim 30, wherein said at least one SARS protein is an S protein and said fragment is Fc.

32. The method of claim 30, wherein said at least one SARS protein is an N protein and said fragment is N195.

33. A diagnostic method for detecting a SARS virus in at least one biological sample, comprising:
(a) contacting said at least one biological sample with at least one monoclonal antibody against a SARS virus protein, and
b) detecting the formation of a complex between said monoclonal antibody and said SARS virus.

34. The diagnostic method of claim 33, wherein said monoclonal antibody is derived from a non-human animal injected with an antigenic fragment of a SARS virus protein.

35. The diagnostic method of claim 33, wherein said monoclonal antibody is derived from a non-human animal injected with
an antigenic peptide comprising at least about 65 contiguous amino acid residues of SEQ ID No. 2, or
an antigenic peptide comprising at least about 65 amino acid residues and having at least about 90% sequence identity with a contiguous number of amino acid residues of SEQ ID No. 2 having about equal length as said antigenic peptide.

36. The diagnostic method of claim 33, wherein said at least one monoclonal antibody is derived from a non-human animal injected with
an antigenic peptide comprising at least about 65 contiguous amino acid residues of SEQ ID No. 4, or
an antigenic peptide comprising at least about 65 amino acid residues and having at least about 90% sequence identity with a contiguous number of amino acid residues of SEQ ID No. 4 having about equal length as said antigenic peptide.

37. The method of claim 33, wherein said antigenic fragment is a fragment of an N or S protein of the SARS virus.

38. The method of claim 37, wherein said antigenic fragment is N195 or Fc.

39. A monoclonal antibody against at least one epitope of a protein of SARS, wherein said at least one epitope is on at least one antigenic fragment of a SARS protein

40. The monoclonal antibody of claim 39, wherein said antigenic fragment is the N195 fragment of the N protein of SARS.

41. The monoclonal antibody of claim 39, wherein said antigenic fragment is the Fc fragment of the S protein of SARS.

42. A recombinant antibody fragment, wherein said recombinant antibody fragment is derived from the monoclonal antibody of claim 39.


SEQUENCE LISTING (25 sequences)

SEQ ID NO: 1 - DNA, 1269 bp, SARS coronavirus
  CDS (1)..(1269)
atgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataa

SEQ ID NO: 2 - protein, 422 aa, SARS coronavirus
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 3 - DNA, 3768 bp, SARS coronavirus
  CDS (1)..(3768)
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagttaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 4 - protein, 1255 aa, SARS coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 5 - DNA, 588 bp, SARS coronavirus
  CDS (1)..(588)
ttgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataa

SEQ ID NO: 6 - protein, 195 aa, SARS coronavirus
LNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 7 - DNA, 684 bp, SARS coronavirus
  CDS (1)..(684)
aggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagt

SEQ ID NO: 8 - protein, 228 aa, SARS coronavirus
RYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRS

SEQ ID NO: 9 - DNA, 29711 bp, SARS coronavirus
tacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgttggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgttgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacatcttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaagactgctcttaagaaatgcaaatctgcattttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttatccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaagagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaattttggcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctgtgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatccgctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagctcttgtatcagacgttggagatagtactgaagtttccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtagtgctgccaagaagaacaacataccttttagactaacttgtgctacaactagacaggttgtcaatgtcataactactaaaatctcactcaagggtggtaagattgttagtacttgttttaaacttatgcttaaggccacattattgtgcgttcttgctgcattggtttgttatatcgttatgccagtacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggtggttcatacaaaaatgacaaaagctgccctgtagtagctgctatcattacaagagagattggtttcatagtgcctggcttaccgggtactgtgctgagagcaatcaatggtgacttcttgcattttctacctcgtgtttttagtgctgttggcaacatttgctacacaccttccaaactcattgagtatagtgattttgctacctctgcttgcgttcttgctgctgagtgtacaatttttaaggatgctatgggcaaacctgtgccatattgttatgacactaatttgctagagggttctatttcttatagtgagcttcgtccagacactcgttatgtgcttatggatggttccatcatacagtttcctaacacttacctggagggttctgttagagtagtaacaacttttgatgctgagtactgtagacatggtacatgcgaaaggtcagaagtaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgtttttggtgagtacaaccatgttgttgctgctaatgcacttttgtttttgatgtctttcactatactctgtctggtaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagccttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaggaggcctatgagcaggctgtagctaatggtgattctgaagtcgttctcaaaaagttaaagaaatctttgaatgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacacagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgccactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggataagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaagttcaagactgaaggattatgtgttgacataccaggcataccaaaggacatgacctaccgtagactcatctctatgatgggtttcaaaatgaattaccaagtcaatggttaccctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgcattgcttgctgataagtttccagttcttcatgacataggaaatccaaaggctatcaagtgtgtgcctcaggctgaagtagaatggaagttctacgatgctcagccatgtagtgacaaagcttacaaaatagaggaactcttctattcttatgctatacatcacgataaattcactgatggtgtttgtttgttttggaattgtaacgttgatcgttacccagccaatgcaattgtgtgtaggtttgacacaagagtcttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttgatgactttgtcgagataataaagtcacaagatttgtcagtgatttcaaaagtggtcaaggttacaattgactatgctgaaatttcattcatgctttggtgtaaggatggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtgctcagacaatggttgccaactggcacactacttgtcgattcagatcttaatgacttcgtctccgacgcagattctactttaattggagactgtgcaacagtacatacggctaataaatgggaccttattattagcgatatgtatgaccctaggaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaacacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaacaactaaacgaacatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagttaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttggatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacactccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacctatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatatgcactgtagtacagcgctgtgcatctaataaacctcatgtgcttgaagatccttgtaaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtacttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgac

SEQ ID NO: 10 - DNA, 31 bp, SARS coronavirus
cgggatccatgtctgataatggaccccaatc

SEQ ID NO: 11 - DNA, 31 bp, SARS coronavirus
acgcgtcgacttatgcctgagttgaatcagc

SEQ ID NO: 12 - DNA, 31 bp, SARS coronavirus; identical to SEQ ID NO: 10

SEQ ID NO: 13 - DNA, 30 bp, SARS coronavirus
acgcgtcgactcgagcaggagaatttcccc

SEQ ID NO: 14 - DNA, 31 bp, SARS coronavirus
cgggatccaaccagcttgagagcaaagtttc

SEQ ID NO: 15 - DNA, 31 bp, SARS coronavirus; identical to SEQ ID NO: 11

SEQ ID NO: 16 - DNA, 29 bp, SARS coronavirus
cgggatccgccttgaatacacccaaagac

SEQ ID NO: 17 - DNA, 30 bp, SARS coronavirus
acgcgtcgacaaattgtgcaatttgcggcc

SEQ ID NO: 18 - DNA, 29 bp, SARS coronavirus
cgggatccgccttgaatacacccaaagac

SEQ ID NO: 19 - DNA, 28 bp, SARS coronavirus
acgcgtcgacagcaggagaatttcccct

SEQ ID NO: 20 - DNA, 29 bp, SARS coronavirus
cgggatccttgaaccagcttgagagcaaa

SEQ ID NO: 21 - DNA, 30 bp, SARS coronavirus; identical to SEQ ID NO: 17

SEQ ID NO: 22 - DNA, 29 bp, SARS coronavirus
cgggatccgatccacaattcaaagacaac

SEQ ID NO: 23 - DNA, 31 bp, SARS coronavirus; identical to SEQ ID NO: 11

SEQ ID NO: 24 - DNA, 32 bp, SARS coronavirus
  misc_feature (3)..(8)
cgggatccaacgtcatactgctgaacaagcac

SEQ ID NO: 25 - DNA, 31 bp, SARS coronavirus; identical to SEQ ID NO: 11
  misc_feature (5)..(10)